(19) United States
(12) Patent Application Publication
(10) Pub. No.: US 2025/0091992 A1
(43) Pub. Date: Mar. 20, 2025

(54) NOVEL COMPOUNDS AND USES THEREOF

(71) Applicants: Ryan Joseph PROTZKO, Marriottsville, MD (US); Thomas PFEIFFER, Greater London (GB); Royston Anthony GRAY, Essex (GB); KANNA HEALTH LIMITED, Kent (GB)

(72) Inventors: Ryan Joseph PROTZKO, Alameda, CA (US); Thomas PFEIFFER, London (GB); Royston Anthony GRAY, Essex (GB)

(21) Appl. No.: 18/581,000

(22) PCT Filed: Jul. 22, 2022
(86) PCT No.: PCT/US2022/074072

§ 371 Date: Jan. 19, 2024

Related U.S. Application Data
(60) Provisional application No. 63/224,470, filed on Jul. 22, 2021.

(51) Int. Cl. C07D 209/12; A61K 31/404; A61P 25/22; A61P 25/24

(52) U.S. Cl. CPC C07D 209/12; A61K 31/404; A61P 25/22; A61P 25/24

(57) ABSTRACT

The present invention relates to a group of synthetic compounds related to the naturally occurring mesembrine alkaloids. The invention further relates to the method of synthesis, the use of these compounds as research tools and their use as pharmaceuticals.

### CROSS-REFERENCE TO RELATED APPLICATIONS

The present application is related to, and claims the benefit of U.S. 63/224,470, filed on 22 Jul. 2021 (22.07.2021), the contents of which are hereby incorporated by reference in their entirety.

### FIELD OF THE INVENTION

The present invention relates to a group of synthetic compounds related to the naturally occurring mesembrine alkaloids. The invention further relates to the method of synthesis, the use of these compounds as research tools and their use as pharmaceuticals.

### BACKGROUND TO THE INVENTION

Mesembrine is an alkaloid which naturally occurs in the Sceletium tortuosum species of plants indigenous to South Africa. The genus Sceletium, classified under the Aizoaceae family, is indigenous to the Western, Eastern and Northern Cape province of South Africa. In addition to mesembrine other alkaloids are found in extracts of Sceletium tortuosum including mesembrenol, Δ7mesembrenone, mesembranol, mesembrenone, and epimesembranol.

Extracts of S. tortuosum have a long history of use in traditional medicine by the San and Khoikhoi people in South Africa where it was used as a masticatory and a medicine to quench their thirst, fight fatigue and for healing, social, and spiritual purposes.

More recently studies have revealed that extracts of the plant have numerous biological properties and extracts of S. tortuosum may be useful in the treatment of anxiety and depression, psychological and psychiatric disorders, improving mood, promoting relaxation and happiness.

An in vivo study in rats demonstrated a positive effect of an extract of S. tortuosum on restraint-induced anxiety (Smith, 2011), and a small series of case reports described preliminary evidence for antidepressant and anxiolytic activity in patients suffering from major depression who were treated with tablets comprising a standardized extract of milled S. tortuosum raw material (Gericke, 2001). A dietary supplement comprising such material is available as Zembrin®.

The mechanisms of action on the central nervous system (CNS) of Zembrin® were identified as the ability to cause blockade of the serotonin (5-HT) transporter and enable selective inhibition of the phosphodiesterase-4 (PDE-4) enzyme (Harvey et al., 2011).

The various alkaloids which occur in S. tortuosum have also been studied in particular the three main alkaloids, mesembrenol, mesembrenone, and mesembrine. All three have been shown to be potently active in a 5-HT transporter binding assay and against PDE-4B activity, (Harvey et al., 2011).

Mesembrenone was described as having a dual activity on 5-HT uptake and PDE-4 inhibition as the difference IC₅₀ concentrations on the two assays was ×17, whereas it was ×258 for mesembrenol and ×5500 mesembrine. However, mesembrine had a greater selectivity for the 5-HT transporter over PDE-4B.

The structure of mesembrine was described by Popelak et al., 1960 and the configuration by P. W. Jeffs et al., 1969. Mesembrine occurs naturally as the (−)-isomer as (−)-mesembrine.

Mesembrine can be isolated from extracts of S. tortuosum, however the content of mesembrine in the plant is relatively low at around 0.3% mesembrine in the leaves and 0.86% in the leaves, stems, and flowers. Alternatively, mesembrine can be synthesized chemically using the method described by Stevens and Wentland, 1968.

Despite the high bioactivity of mesembrine alkaloids at certain molecular targets, these compounds have not been developed into pharmaceutical products due to poor solubility, poor bioavailability, and non-specific molecular interactions of drug targets for treatment of disease.

There is a need for the development of improved mesembrine alkaloid-like compounds, as well as compositions and therapeutic uses thereof.

### BRIEF SUMMARY OF THE INVENTION

At its most general, the present invention relates to novel synthetic mesembrine alkaloids compounds. The synthetic mesembrine alkaloids of the invention have demonstrated inhibitory activity in several in vitro pharmacology models such as the serotonin reuptake transporter (SERT) assay and phosphodiesterase-4 (PDE-4) such as PDE-4B1, PDE-4D2 and PDE-41A assays. They have further been shown to possess anxiolytic activity in in vivo models of depression and anxiety such as the open field test and the forced swim test. In addition, the synthetic mesembrine alkaloids of the invention have shown anti-inflammatory activity in a PBMC model of inflammation.

Therefore, the novel synthetic mesembrine alkaloids of the invention may be useful in the treatment or prevention of medical conditions. More specifically the activity of these compounds is likely to enable their use in the treatment or prevention of medical conditions associated with depression, anxiety, and/or inflammation.

In a first aspect of the present invention there is provided a compound of Formula (I) or a salt thereof,

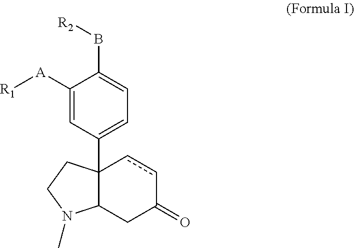

Wherein:
[symbol] is a single or double bond;
A and B are independently O or S;
R¹ and R² are independently selected from the group consisting of:
C₁-C₆ alkyl, C₂-C₆ alkenyl, C₂-C₆ alkynyl, 3- to 8-membered carbocycle, 3- to 8-membered heterocycle, hydrogen, deuterium or halogen, and
wherein A and B are O, R¹ and R² are not mutually a C₁ alkyl or not mutually a hydrogen.

In a second aspect of the present invention, there is provided a compound of Formula (II), or a salt thereof,

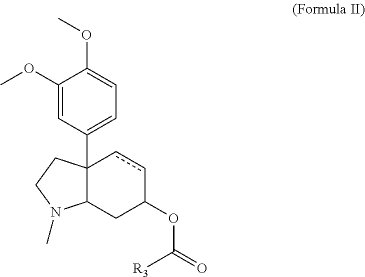

Wherein:
[symbol] is a single or double bond;
R³ is selected from the group consisting of:
C₁-C₆ straight-chain or branched-chain alkyl, C₁-C₆ alkyl carboxylic acid, C₁-C₆ straight chain or branched chain amide, benzyl or pyrrole.

The following compounds are specifically disclaimed from the invention:
rel-(3aR,7aR)-octahydro-3a-[4-methoxy-3-(trifluoromethoxy)phenyl]-1-methyl-6H-indol-6-one;
rel-(3aR,7aR)-3a-[4-(difluoromethoxy)-3-methoxyphenyl]octahydro-1-methyl-6H-indol-6-one;
rel-(3aR,7aR)-octahydro-3a-[4-methoxy-3-(phenylmethoxy)phenyl]-1-methyl-6H-indol-6-one; and
rel-(3aR,7aR)-octahydro-3a-[3-methoxy-4-(phenylmethoxy)phenyl]-1-methyl-6H-indol-6-one.

Preferably the compound of the invention with respect to either Formula (I) or Formula (II) is as defined by any one of the compounds numbered 1 to 61 in Table 1.

In a third aspect of the present invention, there is provided a pharmaceutical composition comprising a compound of Formula I or Formula II, or a salt thereof, together with one or more ingredients selected from carriers, diluents, excipients, adjuvants, fillers, buffers, binders, disintegrants, preservatives, antioxidants, lubricants, stabilisers, solubilisers, surfactants (e.g., wetting agents), masking agents, colouring agents, flavouring agents, and sweetening agents.

In a fourth aspect of the present invention, there is provided a compound of Formula I or Formula II, or the pharmaceutical composition comprising the compound of Formula I or Formula II, for use as a medicament.

In a fifth aspect of the present invention, there is provided a compound of Formula I or Formula II, or the pharmaceutical composition comprising the compound of Formula I or Formula II, for use in the prevention or treatment of disease or condition associated with depression, anxiety, inflammation, and/or autoimmunity.

In preferred embodiments, the prevention or treatment is provided for the group consisting of: addiction, alcoholism, Alzheimer's disease, anxiety, attention deficit disorder (ADHD), binge eating, cluster headaches, complicated grief disorder, depression and anxiety associated with terminal illness, depressive and anxiety disorders, erectile dysfunction, hypersomnia, improving mood in healthy human subjects; insomnia, irritable bowel syndrome, major depression, mania, mental disorders, migraine headaches, pain, panic disorders, Parkinson's disease, post-traumatic mania, post-traumatic stress disorder (PTSD), premature ejaculation, prolonged grief disorder, psychosis, terminal illness, Tourette's syndrome, treatment resistant anxiety, and treatment resistant depression.

In preferred embodiments, the prevention or treatment is provided for the group consisting of: acne vulgaris; acute inflammation; Addison's disease; allergic reactions; allergies; Alzheimer's disease; ankylosing spondylitis; aplastic anemia; asthma; atherosclerosis; autoimmune vasculitis; cancer; celiac disease; chronic inflammatory demyelinating polyneuropathy (CIDP); chronic obstructive pulmonary disease (COPD); colitis; diverticulitis; endometriosis; familial Mediterranean fever; fatty liver disease; glomerulonephritis; Grave's disease; Guillain-Barre syndrome; Hashimoto's thyroiditis; headaches, including chronic headaches and migraine; hemolytic anemia; hidradenitis suppurativa; HIV and AIDS; hypersensitivity reactions; immune-mediated inflammatory disease (IMID); inflammatory bowel disease such as Crohn's disease and ulcerative colitis; inflammatory myopathies; interstitial cystitis; leukocyte defects; lichen planus; mast cell activation syndrome; mastocytosis; mental health conditions where inflammation and/or autoimmunity is a co-morbid or causative factor, including; depression, schizophrenia, and anxiety; multiple sclerosis; myasthenia gravis; obesity; otitis; pain, including acute and chronic pain; Parkinson's disease; pelvic Inflammatory disorder; peripheral ulcerative keratitis; pernicious anemia; pharmacological inflammatory response; pneumonia; prostatitis; psoriasis; psoriatic arthritis; reperfusion injury; rheumatic fever; rheumatoid arthritis; rhinitis; sarcoidosis; scleroderma; Sjogren's syndrome; systemic lupus erythematosus (SLE); transplant rejection syndrome; type I diabetes; type II diabetes; vasculitis; and vitiligo.

In a sixth aspect of the invention, there is a method of treatment comprising administering to a subject in need of treatment a therapeutically effective amount of a compound of Formula I or Formula II.

In a seventh aspect of the invention, there is provided a method of synthesizing the compound of Formula I or Formula II.

In an eight aspect of the invention, there is provided an intermediate formed in the method of synthesis of the compound of Formula I or Formula II.

Preferably the intermediate is a bromine intermediate, as defined by compounds 8 or 9 in Table 1.

These and other aspects and embodiments of the invention are described in further detail below.

### BRIEF SUMMARY OF THE DRAWINGS

The present invention is described with reference to the figures listed below:

FIG. 1 details ¹H NMR of (3aR,7aR)-3a-(3-bromo-4-methoxyphenyl)-1-methyloctahydro-6H-indol-6-one[Compound 6 from Scheme 1 (racemic bromine intermediate)].

FIG. 2 details ¹H NMR of (3aR,7aR)-3a-(3-hydroxy-4-methoxyphenyl)-1-methyloctahydro-6H-indol-6-one[B1-O1-OH from Scheme 2].

FIGS. 3 to 34 detail LCMS traces for the compounds listed in Table 2.

FIG. 35 details the ¹H NMR of Compound 5, deuterated mesembrine.

FIG. 36 details the concentration-effect curves describing functional inhibition of (A) SERT, (B) PDE-4D2 by compounds 11, 13, 21, 24 and 27. 4-parameter logistical fit with variable Hill slope (NH). Each data point represents the mean of two technical replicates at each concentration examined.

FIG. 37 details the time spent in centre of open field test apparatus by mice receiving low dose (0.3 mg/kg) and high dose (3 mg/kg) compounds. Dotted line depicts the time spent in the centre by the vehicle group.

FIG. 38 details the time spent immobile during forced swim test by mice low dose (0.3 mg/kg) and high dose (3 mg/kg) compounds. Dotted line depicts the time spent immobile by the vehicle group.

FIG. 39 details the percentage inhibition of TNF-α in comparison to maximal response to LPS.

FIG. 40 details the percentage inhibition of IL-10 in comparison to maximal response to LPS.

FIG. 41 details the percentage inhibition of IL-β pin comparison to maximal response to LPS.

FIG. 42 details the ratio of IL-10:TNF-α for percentage LPS reduction.

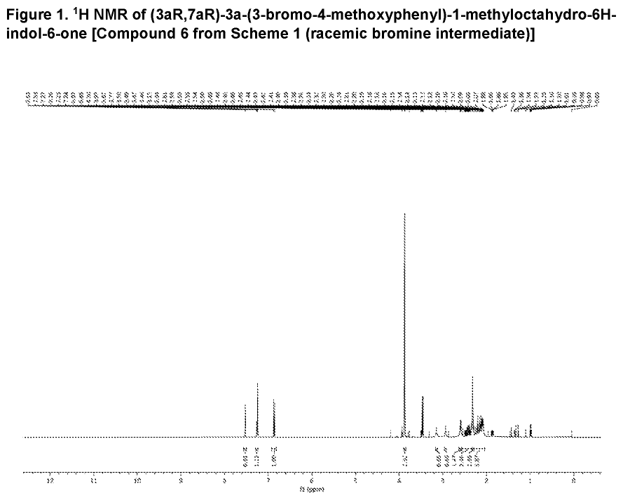

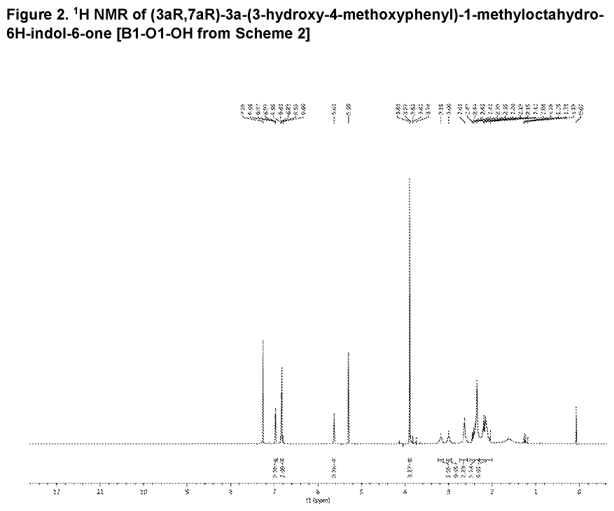

### DEFINITIONS

“Alkyl” refers to substituted or unsubstituted saturated hydrocarbon groups, including straight-chain alkyl and branched-chain alkyl groups. An alkyl group may contain from one to twelve carbon atoms (e.g., C₁₋₁₂ alkyl), such as one to eight carbon atoms (C₁₋₈ alkyl) or one to six carbon atoms (C₁₋₆ alkyl). Exemplary alkyl groups include methyl, ethyl, n-propyl, isopropyl, n-butyl, isobutyl, sec-butyl, tert-butyl, pentyl, isopentyl, neopentyl, hexyl, septyl, octyl, nonyl, and decyl. An alkyl group is attached to the rest of the molecule by a single bond. Unless stated otherwise specifically in the specification, an alkyl group is optionally substituted by one or more substituents such as those substituents described herein.

“Haloalkyl” refers to an alkyl group that is substituted by one or more halogens. Exemplary haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl.

“Alkenyl” refers to substituted or unsubstituted hydrocarbon groups, including straight-chain or branched-chain alkenyl groups containing at least one double bond. An alkenyl group may contain from two to twelve carbon atoms (e.g., C₂₋₁₂ alkenyl). Exemplary alkenyl groups include ethenyl (i.e., vinyl), prop-1-enyl, but-1-enyl, pent-1-enyl, penta-1,4-dienyl, and the like. Unless stated otherwise specifically in the specification, an alkenyl group is optionally substituted by one or more substituents such as those substituents described herein.

“Alkynyl” refers to substituted or unsubstituted hydrocarbon groups, including straight-chain or branched-chain alkynyl groups containing at least one triple bond. An alkynyl group may contain from two to twelve carbon atoms (e.g., C₂₋₁₂ alkynyl). Exemplary alkynyl groups include ethynyl, propynyl, butynyl, pentynyl, hexynyl, and the like. Unless stated otherwise specifically in the specification, an alkynyl group is optionally substituted by one or more substituents such as those substituents described herein.

“Ester” refers to a functional group —COO and may also be referred to as an “ester link”. Esters are formed by the condensation reaction between an alcohol and a carboxylic acid.

“Alkylene” or “alkylene chain” refers to a substituted or unsubstituted saturated, straight or branched divalent hydrocarbon chain. The alkylene group may contain having from 1 to 3 carbon atoms. Non-limiting examples of C₁-C₃ alkylene include methylene, ethylene, and propylene. The alkylene chain is attached to the molecule through a single bond and to a group (e.g., those described herein) through a single bond. The points of attachment of the alkylene chain to the rest of the molecule and to the radical group can be through one carbon or any two carbons within the chain.

“Carbocyclyl,” “carbocyclic ring” or “carbocycle” refers to a substituted or unsubstituted ring structure, wherein the atoms which form the ring are each carbon, and which is attached to the rest of the molecule by a single bond. The carbocycle can be attached to the rest of the molecule through an alkylene group as defined here. Carbocyclic rings can comprise from 3 to 8 carbon atoms in the ring. Carbocyclic rings include aryls and cycloalkyl, cycloalkenyl, and cycloalkynyl as defined herein.

“Cycloalkyl” refers to a stable non aromatic monocyclic or polycyclic fully saturated, substituted or unsubstituted, hydrocarbon consisting solely of carbon and hydrogen atoms, which can include fused or bridged ring systems, having from 3 to 8 carbon atoms and which is attached to the rest of the molecule (optionally through an alkylene group) by a single bond. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7 dimethyl bicyclo[2.2.1]heptanyl, and the like.

“Cycloalkenyl” refers to a stable non aromatic monocyclic or polycyclic, substituted or unsubstituted, hydrocarbon consisting solely of carbon and hydrogen atoms, having one or more carbon-carbon double bonds, which can include fused or bridged ring systems, having from 3 to 8 carbon atoms, and which is attached to the rest of the molecule (optionally through an alkylene group) by a single bond. Monocyclic cycloalkenyls include, for example, cyclopentenyl, cyclohexenyl, cycloheptenyl, cycloctenyl, and the like. Polycyclic cycloalkenyls include, for example, bicyclo[2.2.1]hept-2-enyl and the like.

“Cycloalkynyl” refers to a stable non aromatic monocyclic or polycyclic, substituted or unsubstituted, hydrocarbon consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule (optionally through an alkylene group) by a single bond. Monocyclic cycloalkynyl include, for example, cycloheptynyl, cyclooctynyl, and the like.

“Aryl” refers to a hydrocarbon ring system comprising hydrogen, 6 to 18 carbon atoms and at least one aromatic ring, and which is attached to the rest of the molecule by a single bond. The carbocycle can be attached to the rest of the molecule through an alkylene group as defined here. For purposes of this disclosure, the aryl can be a monocyclic, bicyclic, tricyclic or tetracyclic ring system, which can include fused ring systems. Aryls include, but are not limited to, aryls with six ring carbon atoms e.g., phenyl, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the “aryl” can be optionally substituted.

The term “halo” or, alternatively, “halogen” means fluoro or fluorine, chloro or chlorine, bromo or bromine and iodo or iodine.

“Heterocyclyl,” “heterocyclic ring” or “heterocycle” refers to a stable 3 to 8 membered ring group which consists of 2 to 7 carbon atoms and from 1 to 3 heteroatoms selected from the group consisting of nitrogen, oxygen and sulfur. Heterocycles include heteroaryls as defined below. Unless stated otherwise specifically in the specification, the heterocyclyl group can be a monocyclic, bicyclic, tricyclic or tetracyclic ring system, which can include fused or bridged ring systems; and the nitrogen, carbon or sulfur atoms in the heterocyclyl group can be optionally oxidized; the nitrogen atom can be optionally quaternized; and the heterocyclyl group can be partially or fully saturated. The heterocycles can be attached to the rest of the molecule through an alkylene group as defined here. Examples of such heterocyclyl groups include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2 oxopiperazinyl, 2 oxopiperidinyl, 2 oxopyrrolidinyl, oxazolidinyl, piperidinyl, piperazinyl, 4 piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1 oxo thiomorpholinyl, and 1,1 dioxo thiomorpholinyl. Unless stated otherwise specifically in the specification, a heterocyclyl group can be optionally substituted.

“Heteroaryl” refers to a 5 to 8 membered ring system comprising hydrogen atoms, 1 to 7 carbon atoms, 1 to 3 heteroatoms selected from the group consisting of nitrogen, oxygen and sulfur, at least one aromatic ring, and which is attached to the rest of the molecule by a single bond. For purposes of this disclosure, the heteroaryl can be a monocyclic, bicyclic, tricyclic or tetracyclic ring system, which can include fused or bridged ring systems; and the nitrogen, carbon or sulfur atoms in the heteroaryl can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4 benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2 a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2 oxoazepinyl, oxazolyl, oxiranyl, 1-oxidopyridinyl, 1 oxidopyrimidinyl, 1-oxidopyrazinyl, 1-oxidopyridazinyl, 1 phenyl 1H pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl).

The term “substituted” refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a deuterium, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocycle, an aralkyl, a carbocycle, a heterocycle, a cycloalkyl, a heterocycloalkyl, an aromatic and heteroaromatic moiety.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

Where substituent groups are specified by their conventional chemical formulae, written from left to right, they equally encompass the chemically identical substituents that would result from writing the structure from right to left, e.g., —CH₂O— is equivalent to —OCH₂—.

“Optional” or “optionally” means that the subsequently described event of circumstances may or may not occur, and that the description includes instances where the event or circumstance occurs and instances in which it does not. For example, “optionally substituted aryl” means that the aryl group may or may not be substituted and that the description includes both substituted aryl groups and aryl groups having no substitution.

Compounds of the present disclosure also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Isotopically-enriched compounds may be prepared by conventional techniques well known to those skilled in the art.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents or resolved using conventional techniques. The optical activity of a compound can be analyzed via any suitable method, including but not limited to chiral chromatography and polarimetry, and the degree of predominance of one stereoisomer over the other isomer can be determined.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, chemical entities described herein are intended to include all Z-, E- and tautomeric forms as well.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When stereochemistry is not specified, certain small molecules described herein include, but are not limited to, when possible, their isomers, such as enantiomers and diastereomers, mixtures of enantiomers, including racemates, mixtures of diastereomers, and other mixtures thereof, to the extent they can be made by one of ordinary skill in the art by routine experimentation. In those situations, the single enantiomers or diastereomers, i.e., optically active forms, can be obtained by asymmetric synthesis or by resolution of the racemates or mixtures of diastereomers. Resolution of the racemates or mixtures of diastereomers, if possible, can be accomplished, for example, by conventional methods such as crystallization in the presence of a resolving agent, or chromatography, using, for example, a chiral high-pressure liquid chromatography (HPLC) column. Furthermore, a mixture of two enantiomers enriched in one of the two can be purified to provide further optically enriched form of the major enantiomer by recrystallization and/or trituration. In addition, such certain small molecules include Z- and E-forms (or cis- and trans-forms) of certain small molecules with carbon-carbon double bonds or carbon-nitrogen double bonds. Where certain small molecules described herein exist in various tautomeric forms, the term “certain small molecule” is intended to include all tautomeric forms of the certain small molecule.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In embodiments, the pharmaceutically acceptable base addition salt is chosen from ammonium, potassium, sodium, calcium, and magnesium salts.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. As used herein, “treatment” or “treating” refers to an approach for obtaining beneficial or desired results with respect to a disease, disorder, or medical condition including but not limited to a therapeutic benefit and/or a prophylactic benefit. A therapeutic benefit can include, for example, the eradication or amelioration of the underlying disorder being treated.

Also, a therapeutic benefit can include, for example, the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made.

### DETAILED DESCRIPTION OF THE INVENTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

Modifying mesembrine alkaloids can significantly change their chemical and biological properties. For example, naturally occurring mesembrine and mesembrine alkaloids are scarcely soluble in water, whereas addition of a charged chemical functional group can increase water solubility. Such chemical functional groups can include a polar moiety, a monosaccharide, disaccharide, a carbohydrate, an amino acid, an acyl group, a diacid group, and other chemical moieties. For biological systems, the addition of such modifying functional groups can significantly alter the resulting biological activity or tissue targeting. For the end use of these compounds, modifications have major impacts on downstream formulations, preparations, pharmacokinetics, pharmacodynamics, and ultimate end uses.

### Compounds

In embodiments, the present disclosure provides a compound of Formula (I) or a salt thereof:

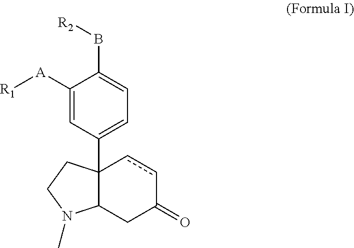

Wherein:
[symbol] is a single or double bond;
A and B are independently O or S;
R¹ and R² are independently selected from the group consisting of:
C₁-Cₑ alkyl, C₂-C₆ alkenyl, C₂-C₆ alkynyl, 3- to 8-membered carbocycle, 3- to 8-membered heterocycle, hydrogen, deuterium or halogen, and
wherein A and B are O, R¹ and R² are not mutually a C₁ alkyl or not mutually a hydrogen.

In embodiments, the present disclosure provides a compound of Formula (II) or a salt thereof:

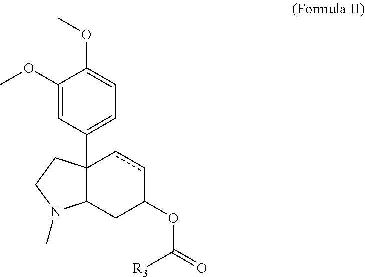

Wherein:
[symbol] is a single or double bond;
R³ is selected from the group consisting of:
C₁-Cₑ straight-chain or branched-chain alkyl, C₁-Cₑ alkyl carboxylic acid, C₁-Cₑ straight chain or branched chain amide, benzyl or pyrrole.

In embodiments of the compounds of Formula (I) the compound is not:

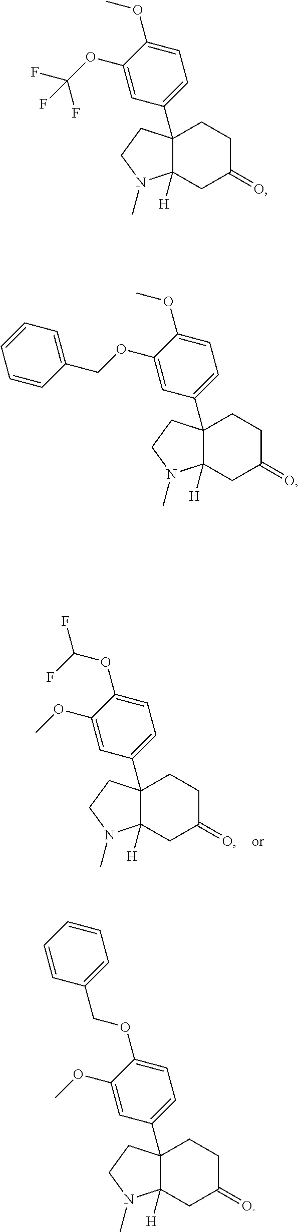 [SMILES: COc1ccc(C23CCC(=O)CC2N(C)CC3)cc1OC(F)(F)F ; COc1ccc(C23CCC(=O)CC2N(C)CC3)cc1OCc1ccccc1 ; COc1cc(C23CCC(=O)CC2N(C)CC3)ccc1OC(F)F ; COc1cc(C23CCC(=O)CC2N(C)CC3)ccc1OCc1ccccc1]

In embodiments of the compounds of Formula (I), the compound is not:

[symbol]

In a further embodiment the compound of Formula (I) is:

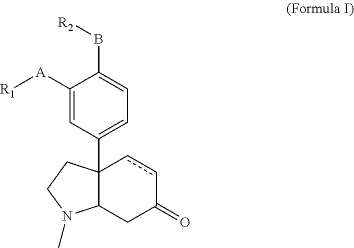

Wherein:
[symbol] is a single or double bond;
A and B are O;
R¹ and R² are independently selected from the group consisting of:
C₁-Cₑ alkyl, C₂-C₆ alkenyl, C₂-C₆ alkynyl, 3- to 8-membered carbocycle, 3- to 8-membered heterocycle, hydrogen, deuterium or halogen, and
wherein R¹ and R² are not mutually a C₁ alkyl or not mutually a hydrogen.

In a further embodiment the compound of Formula (I) is:

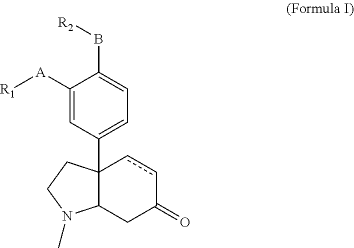

Wherein:
[symbol] is a single or double bond;
A and B are S;
R¹ and R² are independently selected from the group consisting of:
C₁-Cₑ alkyl, C₂-C₆ alkenyl, C₂-C₆ alkynyl, 3- to 8-membered carbocycle, 3- to 8-membered heterocycle, hydrogen, deuterium or halogen.

In a further embodiment the compound of Formula (I) is

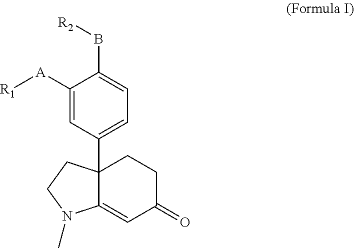

Wherein:
A and B are independently O or S;
R¹ and R² are independently selected from the group consisting of:
C₁-Cₑ alkyl, C₂-C₆ alkenyl, C₂-C₆ alkynyl, 3- to 8-membered carbocycle, 3- to 8-membered heterocycle, hydrogen, deuterium or halogen.

In embodiments, provided herein is one or more compounds selected from Table 1.

In embodiments, provided herein is one or more pharmaceutically acceptable salts of a compound selected from Table 1.

TABLE 1
Compounds

| | |
|---|---|
| 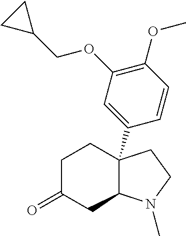 [SMILES: COc1ccc([C@@]23CCC(=O)C[C@@H]2N(C)CC3)cc1OCC1CC1] | 1 |
| 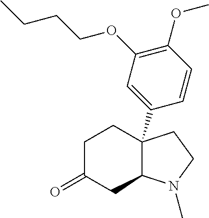 [SMILES: CCCCOc1cc([C@@]23CCC(=O)C[C@@H]2N(C)CC3)ccc1OC] | 2 |
| 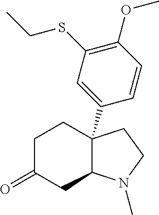 [SMILES: CCSc1cc([C@@]23CCC(=O)C[C@@H]2N(C)CC3)ccc1OC] | 3 |
| 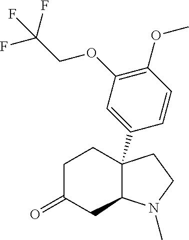 [SMILES: COc1ccc([C@@]23CCC(=O)C[C@@H]2N(C)CC3)cc1OCC(F)(F)F] | 4 |
| 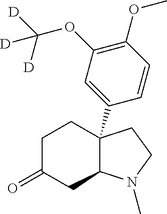 [SMILES: [2H]C([2H])([2H])Oc1cc([C@@]23CCC(=O)C[C@@H]2N(C)CC3)ccc1OC] | 5 |
| 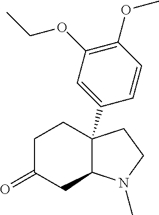 [SMILES: CCOc1cc([C@@]23CCC(=O)C[C@@H]2N(C)CC3)ccc1OC] | 6 |
| 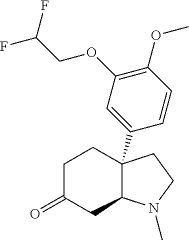 [SMILES: COc1ccc([C@@]23CCC(=O)C[C@@H]2N(C)CC3)cc1OCC(F)F] | 7 |
| 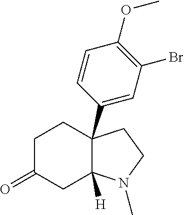 [SMILES: COc1ccc([C@]23CCC(=O)C[C@H]2N(C)CC3)cc1Br] | 8 |
| 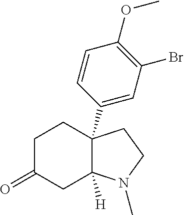 [SMILES: COc1ccc([C@@]23CCC(=O)C[C@@H]2N(C)CC3)cc1Br] | 9 |
| 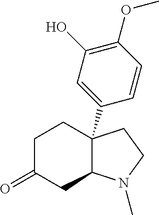 [SMILES: COc1ccc([C@@]23CCC(=O)C[C@@H]2N(C)CC3)cc1O] | 10 |
| 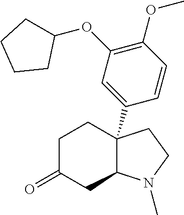 [SMILES: COc1ccc([C@@]23CCC(=O)C[C@@H]2N(C)CC3)cc1OC1CCCC1] | 11 |
| 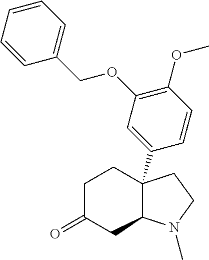 [SMILES: COc1ccc([C@@]23CCC(=O)C[C@@H]2N(C)CC3)cc1OCc1ccccc1] | 12 |
| 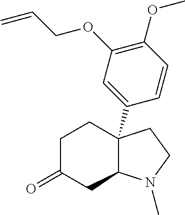 [SMILES: C=CCOc1cc([C@@]23CCC(=O)C[C@@H]2N(C)CC3)ccc1OC] | 13 |
| 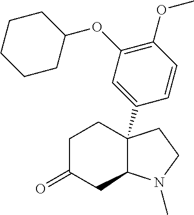 [SMILES: COc1ccc([C@@]23CCC(=O)C[C@@H]2N(C)CC3)cc1OC1CCCCC1] | 14 |
| 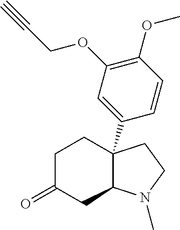 [SMILES: C#CCOc1cc([C@@]23CCC(=O)C[C@@H]2N(C)CC3)ccc1OC] | 15 |
| 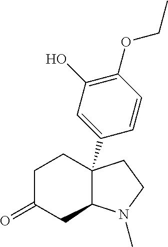 [SMILES: CCOc1ccc([C@@]23CCC(=O)C[C@@H]2N(C)CC3)cc1O] | 16 |
| 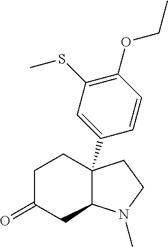 [SMILES: CCOc1ccc([C@@]23CCC(=O)C[C@@H]2N(C)CC3)cc1SC] | 17 |
| 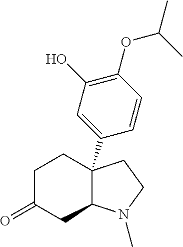 [SMILES: CC(C)Oc1ccc([C@@]23CCC(=O)C[C@@H]2N(C)CC3)cc1O] | 18 |
| 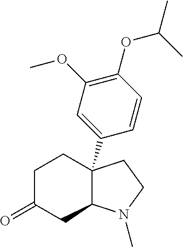 [SMILES: COc1cc([C@@]23CCC(=O)C[C@@H]2N(C)CC3)ccc1OC(C)C] | 19 |
| 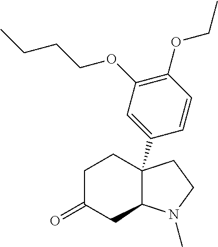 [SMILES: CCCCOc1cc([C@@]23CCC(=O)C[C@@H]2N(C)CC3)ccc1OCC] | 20 |
| 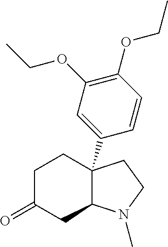 [SMILES: CCOc1ccc([C@@]23CCC(=O)C[C@@H]2N(C)CC3)cc1OCC] | 21 |
| 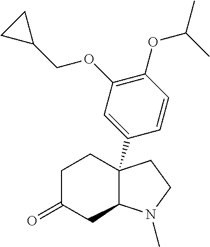 [SMILES: CC(C)Oc1ccc([C@@]23CCC(=O)C[C@@H]2N(C)CC3)cc1OCC1CC1] | 22 |
| 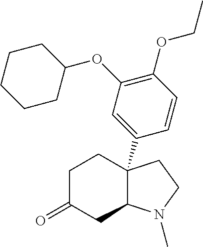 [SMILES: CCOc1ccc([C@@]23CCC(=O)C[C@@H]2N(C)CC3)cc1OC1CCCCC1] | 23 |
| 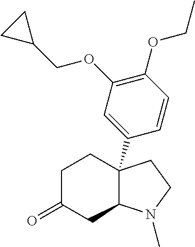 [SMILES: CCOc1ccc([C@@]23CCC(=O)C[C@@H]2N(C)CC3)cc1OCC1CC1] | 24 |
| 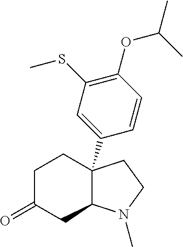 [SMILES: CSc1cc([C@@]23CCC(=O)C[C@@H]2N(C)CC3)ccc1OC(C)C] | 25 |
| 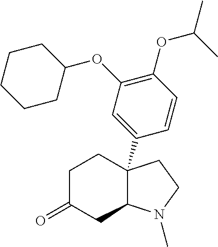 [SMILES: CC(C)Oc1ccc([C@@]23CCC(=O)C[C@@H]2N(C)CC3)cc1OC1CCCCC1] | 26 |
| 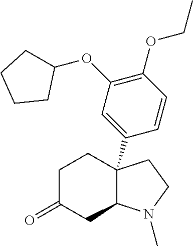 [SMILES: CCOc1ccc([C@@]23CCC(=O)C[C@@H]2N(C)CC3)cc1OC1CCCC1] | 27 |
| 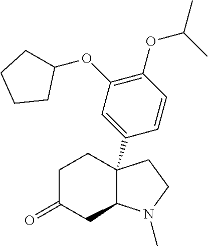 [SMILES: CC(C)Oc1ccc([C@@]23CCC(=O)C[C@@H]2N(C)CC3)cc1OC1CCCC1] | 28 |
| 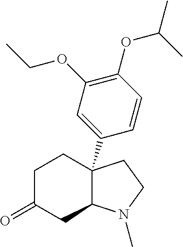 [SMILES: CCOc1cc([C@@]23CCC(=O)C[C@@H]2N(C)CC3)ccc1OC(C)C] | 29 |
| 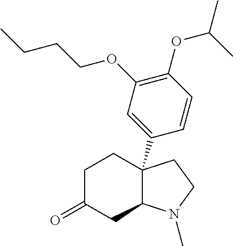 [SMILES: CCCCOc1cc([C@@]23CCC(=O)C[C@@H]2N(C)CC3)ccc1OC(C)C] | 30 |
| 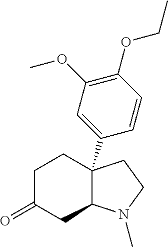 [SMILES: CCOc1ccc([C@@]23CCC(=O)C[C@@H]2N(C)CC3)cc1OC] | 31 |
| 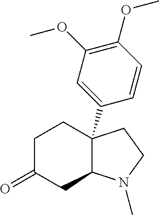 [SMILES: COc1ccc([C@@]23CCC(=O)C[C@@H]2N(C)CC3)cc1OC] | 32 |
| 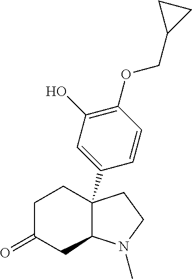 [SMILES: CN1CC[C@]2(c3ccc(OCC4CC4)c(O)c3)CCC(=O)C[C@H]12] | 33 |
| 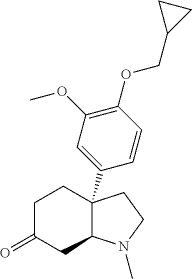 [SMILES: COc1cc([C@@]23CCC(=O)C[C@@H]2N(C)CC3)ccc1OCC1CC1] | 34 |
| 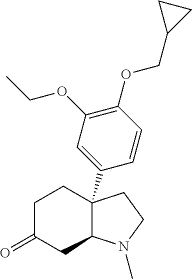 [SMILES: CCOc1cc([C@@]23CCC(=O)C[C@@H]2N(C)CC3)ccc1OCC1CC1] | 35 |
| 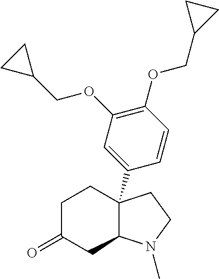 [SMILES: CN1CC[C@]2(c3ccc(OCC4CC4)c(OCC4CC4)c3)CCC(=O)C[C@H]12] | 36 |
| 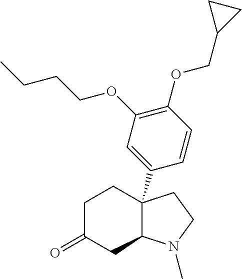 [SMILES: CCCCOc1cc([C@@]23CCC(=O)C[C@@H]2N(C)CC3)ccc1OCC1CC1] | 37 |
| 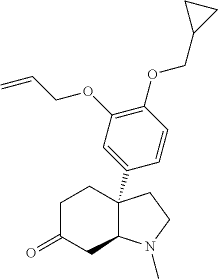 [SMILES: C=CCOc1cc([C@@]23CCC(=O)C[C@@H]2N(C)CC3)ccc1OCC1CC1] | 38 |
| 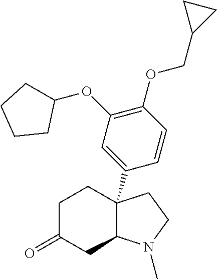 [SMILES: CN1CC[C@]2(c3ccc(OCC4CC4)c(OC4CCCC4)c3)CCC(=O)C[C@H]12] | 39 |
| 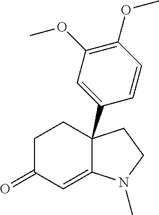 [SMILES: COc1ccc([C@]23CCC(=O)C=C2N(C)CC3)cc1OC] | 40 |
| 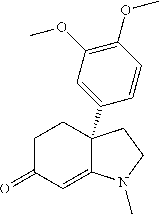 [SMILES: COc1ccc([C@@]23CCC(=O)C=C2N(C)CC3)cc1OC] | 41 |
| 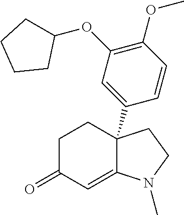 [SMILES: COc1ccc([C@@]23CCC(=O)C=C2N(C)CC3)cc1OC1CCCC1] | 42 |
| 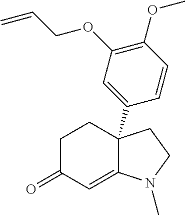 [SMILES: C=CCOc1cc([C@@]23CCC(=O)C=C2N(C)CC3)ccc1OC] | 43 |
| 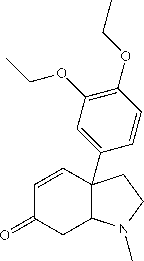 [SMILES: CCOc1ccc(C23C=CC(=O)CC2N(C)CC3)cc1OCC] | 44 |
| 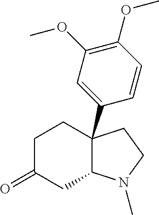 [SMILES: COc1ccc([C@]23CCC(=O)C[C@H]2N(C)CC3)cc1OC] | 45 |
| 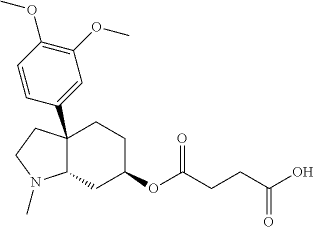 [SMILES: COc1ccc([C@@]23CC[C@@H](OC(=O)CCC(=O)O)C[C@@H]2N(C)CC3)cc1OC] | 46 |
| 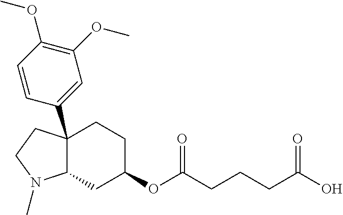 [SMILES: COc1ccc([C@@]23CC[C@@H](OC(=O)CCCC(=O)O)C[C@@H]2N(C)CC3)cc1OC] | 47 |
| 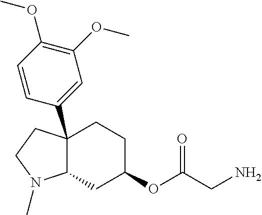 [SMILES: COc1ccc([C@@]23CC[C@@H](OC(=O)CN)C[C@@H]2N(C)CC3)cc1OC] | 48 |
| 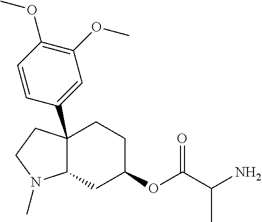 [SMILES: COc1ccc([C@@]23CC[C@@H](OC(=O)C(C)N)C[C@@H]2N(C)CC3)cc1OC] | 49 |
| 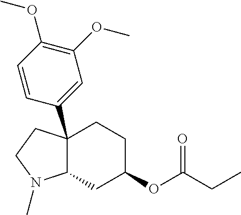 [SMILES: CCC(=O)O[C@@H]1CC[C@@]2(c3ccc(OC)c(OC)c3)CCN(C)[C@H]2C1] | 50 |
| 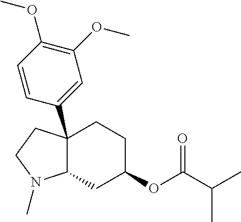 [SMILES: COc1ccc([C@@]23CC[C@@H](OC(=O)C(C)C)C[C@@H]2N(C)CC3)cc1OC] | 51 |
| 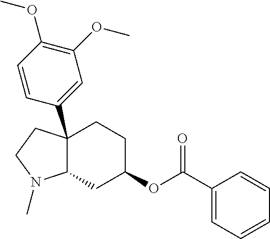 [SMILES: COc1ccc([C@@]23CC[C@@H](OC(=O)c4ccccc4)C[C@@H]2N(C)CC3)cc1OC] | 52 |
| 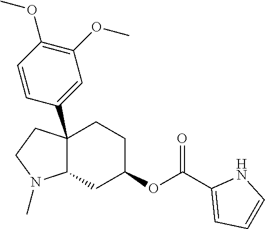 [SMILES: COc1ccc([C@@]23CC[C@@H](OC(=O)c4ccc[nH]4)C[C@@H]2N(C)CC3)cc1OC] | 53 |
| 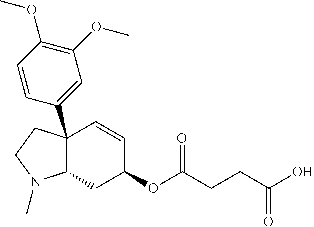 [SMILES: COc1ccc([C@@]23C=C[C@@H](OC(=O)CCC(=O)O)C[C@@H]2N(C)CC3)cc1OC] | 54 |
| 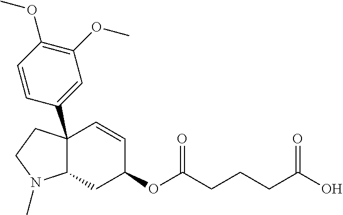 [SMILES: COc1ccc([C@@]23C=C[C@@H](OC(=O)CCCC(=O)O)C[C@@H]2N(C)CC3)cc1OC] | 55 |
| 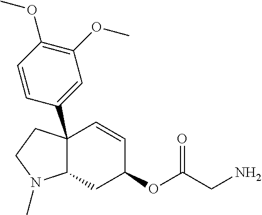 [SMILES: COc1ccc([C@@]23C=C[C@@H](OC(=O)CN)C[C@@H]2N(C)CC3)cc1OC] | 56 |
| 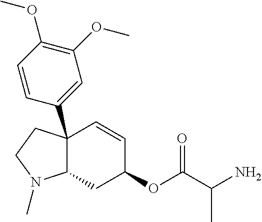 [SMILES: COc1ccc([C@@]23C=C[C@@H](OC(=O)C(C)N)C[C@@H]2N(C)CC3)cc1OC] | 57 |
| 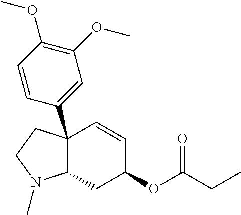 [SMILES: CCC(=O)O[C@@H]1C=C[C@@]2(c3ccc(OC)c(OC)c3)CCN(C)[C@H]2C1] | 58 |
| 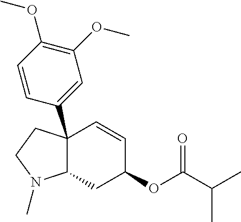 [SMILES: COc1ccc([C@@]23C=C[C@@H](OC(=O)C(C)C)C[C@@H]2N(C)CC3)cc1OC] | 59 |
| 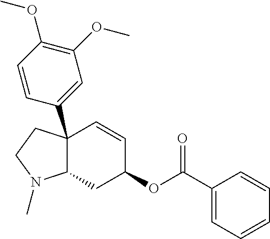 [SMILES: COc1ccc([C@@]23C=C[C@@H](OC(=O)c4ccccc4)C[C@@H]2N(C)CC3)cc1OC] | 60 |
| 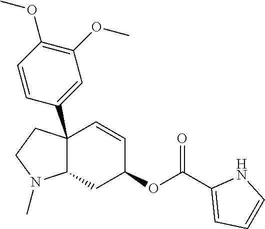 [SMILES: COc1ccc([C@@]23C=C[C@@H](OC(=O)c4ccc[nH]4)C[C@@H]2N(C)CC3)cc1OC] | 61 |

### Compositions

The compounds described herein may be formulated as a pharmaceutical composition. A pharmaceutical composition may comprise: (i) a compound of Formula (I) or Formula (II), as detailed in Table 1 or a pharmaceutically acceptable salt thereof; and (ii) a pharmaceutically acceptable carrier.

In embodiments, a pharmaceutically acceptable carrier includes a pharmaceutically acceptable excipient, binder, and/or diluent. In embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, hydroxymethylcellulose and polyvinylpyrrolidone. In embodiments, suitable pharmaceutically acceptable carriers include, but are not limited to, inert solid fillers or diluents and sterile aqueous or organic solutions. Sterile phosphate-buffered saline is one example of a pharmaceutically acceptable carrier. Other suitable carriers, diluents, or excipients are well-known to those in the art. (See, e.g., Gennaro (ed.), Remington's Pharmaceutical Sciences (Mack Publishing Company, 19th ed. 1995).) Formulations can further include one or more excipients, preservatives, solubilizers, buffering agents, albumin to prevent protein loss on vial surfaces, etc.

Pharmaceutical compositions comprising a compound of Formula (I) or Formula (II), as detailed in Table 1 or a pharmaceutically acceptable salt thereof, may also contain one or more additional ingredients including, but not limited to, a mucoadhesive compound, a buffering agent, a plasticizing agent, a stabilizing agent, a taste-masking agent, a flavoring agent, a coloring agent, an antiseptic, an inert filler agent, a preservative, and combinations thereof.

In embodiments, the formulations may comprise one or more solubilizing agents that increase the solubility of active compounds in the formulation. Suitable solubilizing agents include, for example, complexing agents, surfactants, and the like. Suitable complexing agents include unsubstituted cyclodextrins (such as alpha-cyclodextrin, beta-cyclodextrin) and substituted cyclodextrins, (such as hydroxypropyl beta-cyclodextrin, sulfobutylether-beta-cyclodextrin). Suitable surfactants include polyoxyethylene sorbitan monolaurate (for example, Tween 20), polyoxyethylene sorbitans molooleate (for example, Tween 80), polyethylene glycol (15)-hydroxystearate (for example, Kolliphor® HS 15), PEG-35 castor oil (for example, Kolliphor® EL) and PEG-60 hydrogenated castor oil (for example, Cremophor® RH 60).

In embodiments, the formulations comprise one or more buffer agents that maintain the pH of the IV solution within a pharmaceutically acceptable range. In certain embodiments, the buffer maintains the pH of the IV solution between about 5 and 9. In specific embodiments, the buffer maintains the pH of the IV solution at about 7.4. Suitable buffers include, for example, citrates, lactate, acetate, maleate, phosphates, and the like. In embodiments, the formulations comprise one or more density modifiers that is used to control the density of the IV formulation. Suitable density modifiers include, for example, dextrose. In embodiments, the formulations comprise one or more isotonicity modifiers that provide a formulation that is iso-osmotic with tissue to prevent pain and irritation when the formulation is administered. Suitable isotonicity modifiers include, for example, electrolytes, monosaccharides, and disaccharides. Examples of isotonicity modifiers include glycerin, dextrose, potassium chloride, and sodium chloride.

In embodiments, the formulations comprise one or more viscosity enhancers. Suitable viscosity enhancers include, for example, povidone, hydroxyethylcellulose, polyvinyl alcohol, and carbomer (such as, acrylic acid homopolymers and acrylic acid copolymers).

In embodiments, the formulations comprise one or more preservatives that increase the stability of active compounds in the formulation and/or provide antimicrobial activity. Suitable preservatives include, for example, antimicrobial agents and antioxidants. Examples of antimicrobial agents include benzyl alcohol, methyl paraben, propyl paraben, phenol, cresol, methyl paraben, chlorbutanol, sodium metabisulphite, sodium bisulphite, benzethonium chloride, and benzalkonium chloride. Examples of antioxidants include sodium bisulphite and other sulfurous acid salts, ascorbic acid, salts of ethylenediaminetetraacetic acid (including sodium), alpha tocopherol, butylated hydroxyl hydroxytoluene, and butylated hydroxyanisole.

A pharmaceutical composition comprising a compound of Formula (I) or Formula (II), as detailed in Table 1, or a pharmaceutically acceptable salt thereof; may be formulated in a dosage form selected from the group consisting of: an oral unit dosage form, an intravenous unit dosage form, an intranasal unit dosage form, a suppository unit dosage form, an intradermal unit dosage form, an intramuscular unit dosage form, an intraperitoneal unit dosage form, a subcutaneous unit dosage form, an epidural unit dosage form, a sublingual unit dosage form, a liquid, a lozenge, a fast disintegrating tablet, a lyophilized preparation, a film, a spray (including a nasal spray, an oral spray, or a topical spray), or a mucoadhesive. The oral unit dosage form may be selected from the group consisting of: tablets, pills, pellets, capsules, powders, lozenges, granules, solutions, suspensions, emulsions, syrups, elixirs, sustained-release formulations, aerosols, and sprays. In embodiments, the modified mesembrine alkaloid is formulated as a liquid, a lozenge, a fast-disintegrating tablet, a lyophilized preparation, a film, a spray, or a mucoadhesive.

The compounds of Formula (I) or Formula (II), as detailed in Table 1, or a pharmaceutically acceptable salt thereof can be administered to subjects by a variety of administration modes, including, for example, by intramuscular, subcutaneous, intravenous, intra-atrial, intra-articular, parenteral, intranasal, intrapulmonary, transdermal, intrapleural, intrathecal, and oral routes of administration. For prevention and treatment purposes, a compound of Formula (I) or Formula (II), as detailed in Table 1, or a pharmaceutically acceptable salt thereof can be administered to a subject in a single bolus delivery, via continuous delivery (e.g., continuous transdermal delivery) over an extended time period, or in a repeated administration protocol (e.g., on an hourly, daily, weekly, or monthly basis).

Pharmaceutical compositions comprising a compound of Formula (I) or Formula (II), as detailed in Table 1, or a pharmaceutically acceptable salt thereof can be supplied as a kit comprising a container that comprises the pharmaceutical composition as described herein. A pharmaceutical composition can be provided, for example, in the form of an injectable solution for single or multiple doses, or as a sterile powder that will be reconstituted before injection. Alternatively, such a kit can include a dry-powder disperser, liquid aerosol generator, or nebulizer for administration of a pharmaceutical composition. Such a kit can further comprise written information on indications and usage of the pharmaceutical composition.

### Therapeutic Use

The compounds of Formula (I) or Formula (II), as detailed in Table 1, have demonstrated inhibitory activity in several in vitro pharmacology models such as a serotonin reuptake transporter (SERT) assay and phosphodiesterase-4 (PDE-4) such as PDE-4B1, PDE-4D2 and PDE-41A assays. They have further been shown to possess anxiolytic activity in in vivo models of depression and anxiety such as the open field test and the forced swim test. In addition, the synthetic mesembrine alkaloids of the invention have shown anti-inflammatory activity in a PBMC model on inflammation.

Therefore, the novel synthetic mesembrine alkaloids of the invention may be useful in the treatment or prevention of medical conditions. More specifically the activity of these compounds is likely to enable their use in the treatment or prevention of medical conditions associated with depression, anxiety, and/or inflammation.

For example, in embodiments compounds of Formula (I) or Formula (II), as detailed in Table 1 are useful in methods for modulating serotonin reuptake transporter (SERT) activity or phosphodiesterase-4 (PDE-4) activity in a subject in need thereof.

Accordingly, in embodiments the present disclosure provides the use of the compounds of Formula (I) or Formula (II), as detailed in Table 1 or a pharmaceutically acceptable salt thereof, for modulating SERT activity. In embodiments, the modulating serotonin reuptake transporter activity is inhibiting serotonin reuptake transporter activity.

Accordingly, in embodiments the present disclosure provides the use of the compounds of Formula (I) or Formula (II), as detailed in Table 1 or a pharmaceutically acceptable salt thereof, for modulating PDE-4 activity in a subject in need thereof. In embodiments, the modulating phosphodiesterase activity is inhibiting PDE-4 activity.

In embodiments, the isoform of PDE-4 is PDE-4A, PDE-4B, PDE-4C or PDE-4D. In embodiments, one or more isoforms of PDE-4 is inhibited e.g., PDE-4A, PDE-4B, PDE-4C and/or PDE-4D.

In embodiments, the compounds of Formula (I) or Formula (II), as detailed in Table 1 or a pharmaceutically acceptable salt thereof are for use in the treatment of the group consisting of: addiction, alcoholism, Alzheimer's disease, anxiety, attention deficit disorder (ADHD), binge eating, cluster headaches, complicated grief disorder, depression and anxiety associated with terminal illness, depressive and anxiety disorders, erectile dysfunction, hypersomnia, improving mood in healthy human subjects; insomnia, irritable bowel syndrome, major depression, mania, mental disorders, migraine headaches, pain, panic disorders, Parkinson's disease, post-traumatic mania, post-traumatic stress disorder (PTSD), premature ejaculation, prolonged grief disorder, psychosis, terminal illness, Tourette's syndrome, treatment resistant anxiety, and treatment resistant depression.

In embodiments, the compounds of Formula (I) or Formula (II), as detailed in Table 1 or a pharmaceutically acceptable salt thereof are for use in the treatment of the group consisting of: acne vulgaris; acute inflammation; Addison's disease; allergic reactions; allergies; Alzheimer's disease; ankylosing spondylitis; aplastic anemia; asthma; atherosclerosis; autoimmune vasculitis; cancer; celiac disease; chronic inflammatory demyelinating polyneuropathy (CIDP); chronic obstructive pulmonary disease (COPD); colitis; diverticulitis; endometriosis; familial Mediterranean fever; fatty liver disease; glomerulonephritis; Grave's disease; Guillain-Barre syndrome; Hashimoto's thyroiditis; headaches, including chronic headaches and migraine; hemolytic anemia; hidradenitis suppurativa; HIV and AIDS; hypersensitivity reactions; immune-mediated inflammatory disease (IMID); inflammatory bowel disease such as Crohn's disease and ulcerative colitis; inflammatory myopathies; interstitial cystitis; leukocyte defects; lichen planus; mast cell activation syndrome; mastocytosis; mental health conditions where inflammation and/or autoimmunity is a co-morbid or causative factor, including; depression, schizophrenia, and anxiety; multiple sclerosis; myasthenia gravis; obesity; otitis; pain, including acute and chronic pain; Parkinson's disease; pelvic Inflammatory disorder; peripheral ulcerative keratitis; pernicious anemia; pharmacological inflammatory response; pneumonia; prostatitis; psoriasis; psoriatic arthritis; reperfusion injury; rheumatic fever; rheumatoid arthritis; rhinitis; sarcoidosis; scleroderma; Sjogren's syndrome; systemic lupus erythematosus (SLE); transplant rejection syndrome; type I diabetes; type II diabetes; vasculitis; and vitiligo.

### Worked Examples

Compounds of the present invention were synthesized using the methods described in Examples 1 to 3.

Reaction products can be purified by known methods including silica gel chromatography using various organic solvents such as hexane, dichloromethane, ethyl acetate, methanol and the like or preparative reverse phase high pressure liquid chromatography.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene and Wuts, Protective Groups in Organic Synthesis, 44th. Ed., Wiley & Sons, 2006, as well as in Jerry March, Advanced Organic Chemistry, 4th edition, John Wiley & Sons, publisher, New York, 1992 which are incorporated herein by reference in their entirety.

The examples below detail the synthetic methods used to prepare the compounds of the invention, namely compounds of Formula (I) and Formula (II). In addition, Example 1 details the preparation of a novel intermediate compound which is the building block required to make the compounds of Formula (I).

The compounds of Formula (I) were synthesized as described in Example 2. Generally, the compounds were synthesized in mg quantities at high purity (>90%) but for several of the compounds which were to be used in the in vivo efficacy studies quantities of over 100 mg were produced, demonstrating the efficacy of the methodology.

The compounds of Formula (II) were synthesized as described in Example 3.

Examples 4 to 10 describe the efficacy of the compounds in several in vitro and in vivo models. The compounds tested were illustrative of the compounds of the invention and as such provide robust evidence that these compounds are useful agents in the treatment of various disease paradigms.

### Example 1: Preparation of the Bromine Intermediate

In order to prepare the compounds of Formula (I), several synthetic steps were required. The starting scheme was the preparation of the bromine intermediate or building block 1. This compound is equivalent to compound identification numbers 8 or 9 from Table 1, depending on the stereochemistry of the compound.

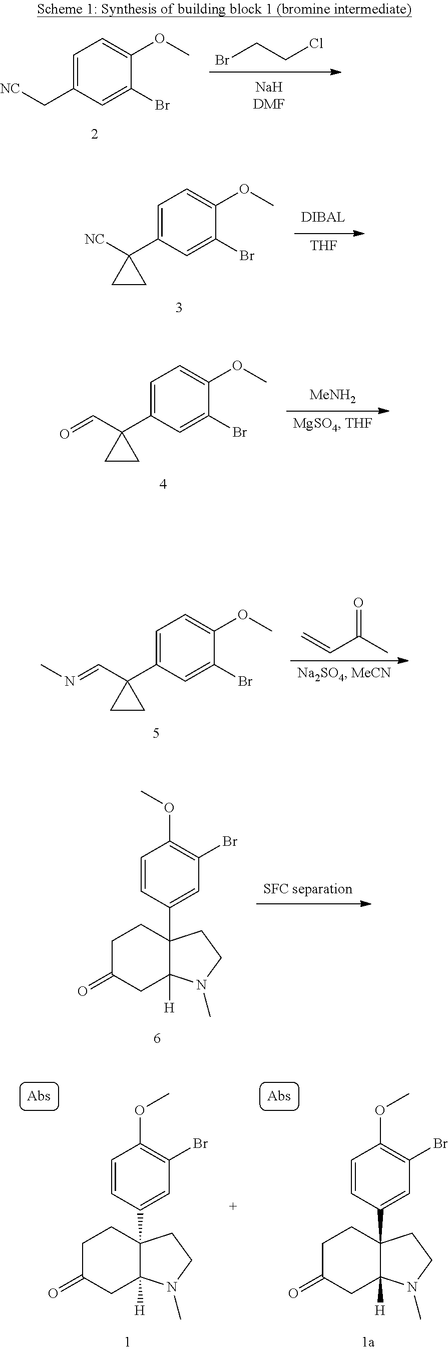

Scheme 1 above details the preparation of the bromine intermediate (6). In detail, acetonitrile 2 was converted to 3 using NaH and DMF, giving yields up to 96%. This could be converted to aldehyde 4 with DIBAL, with yields up to 83%. Aldehyde 4 was converted into imine 5 with quantitative yields.

As imines are sensitive to hydrolysis, this had to be used immediately for the next step. The imine was treated with HCl to convert it to an un-isolated enamine intermediate, which was reacted directly with MVK in the presence of Na₂SO₄. The yield of this varied around 47-61% on the gram scale reactions.

The material was then purified by chiral SFC to isolate the two enantiomers. Bromide 1 was synthesized from 3 scales of 2 to 6 (0.5 g, 5.0 g and 13.4 g of 2), and two SFCs, giving 4.4 g of 1 in total. The total yield of 2 to 1 was 16%.

Compound 6 is a racemate, and the first SFC fraction (enantiomer 1) were converted to mesembrine by Buchwald coupling. A UPC method was developed with the racemic mesembrine, and the enantiopure mesembrine was compared with a natural extract of (−)-Mesembrine. As the peaks matched, it was determined that the first SFC fraction was enantiomer 1 and not 1a.

### Example 2: Preparation of the Compounds of Formula (I)

After successfully preparing the bromine intermediate a synthetic route was devised to prepare compounds where the R1 group was substituted. Scheme 2 below details the alternative routes that were used depending on the final compound formed.

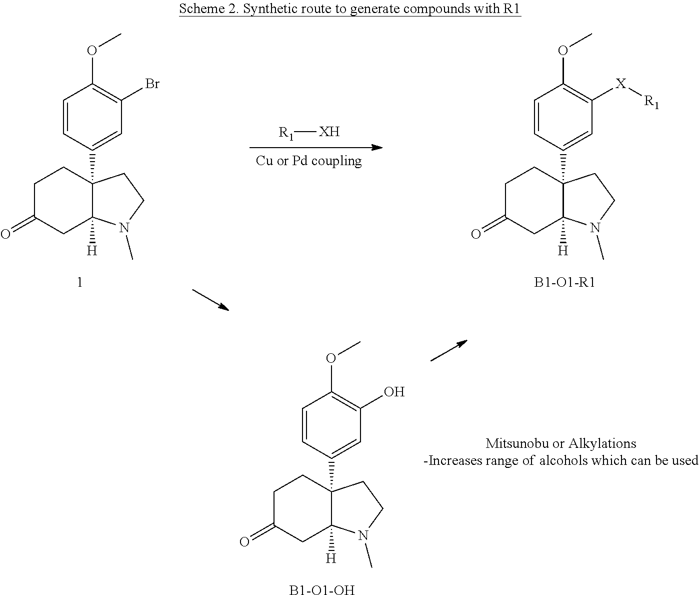

With 6 (rac-1), several organometallic catalytic coupling conditions were trialed to form mesembrine. Ullmann and Buchwald coupling conditions were trialed and Buchwald coupling was chosen as the preferred conditions. The Buchwald coupling of sulfides was also developed.

The general conditions for compound formation are as described below. Table 2 details the compounds that were generated and the coupling conditions used with reference to the compound identity number in Table 1

A: Buchwald coupling for alcohols: Di-tert-butyl(2′,4′,6′-triisopropyl-3,6-dimethoxy-[1,1′-biphenyl]-2-yl)phosphane (0.054 mmol) and Pd₂dba₃ (0.027 eq) were added to a sealed vessel, then a solution of the mesembrine-bromide in toluene (1 eq, 167 g/L) was added, followed by the alcohol (3-4 eq). The reaction was heated to 70° C. If the reaction was incomplete over 16 hours, the heat was raised to 80° C. and then 90° C. Once the reaction was complete the reaction was filtered over C18 endcapped cartridge and washed with DMF and purified by preparative HPLC.

B: Buchwald coupling for sulfides: {2-[2-(diphenylphosphanyl)phenoxy]phenyl}diphenylphosphane (0.2-0.3 mmol), Pd₂dba₃ (0.1-0.15 eq), K₂CO₃ (1.3-1.4 eq) and sodium thioalkoxide (1.2-1.4 eq) were added to a sealed vessel, then a solution of the mesembrine-bromide in toluene (1 eq, 167 g/L) was added, followed by the alcohol (3-4 eq). The reaction was heated to 110° C. for 20 hours. Once the reaction was complete the reaction was filtered over C18 endcapped cartridge and washed with DMF and purified by preparative HPLC.

C: Alkylation: A solution of mesembrine-phenol in DMF (1 eq, 50 g/L) was added to K₂CO₃ (2 eq), followed by the alkylhalide (2.1 eq) under N₂. The reaction could be heated to 50° C. and would usually reach completion within 6 hours. If there was a methyl-cyclopropyl group present, the reaction should be kept at 20-30° C. to minimize side reactions. Once the reaction was complete the reaction was filtered and washed with DMF and purified by preparative HPLC.

D: Mitsunobu reaction: A solution of mesembrine phenol in THF (1 eq, 50 g/L) was added to PPh₃ (1.5 eq), followed by the alcohol (1.5 eq), then DIAD (1.5 eq). The reaction was left overnight at room temperature under N₂. If the reaction was still incomplete, additional alcohol, DIAD and PPh₃ was added until HPLC suggested the reaction was complete. Once complete the solvent was removed by evaporation and redissolved in DMSO and purified by preparative HPLC.

Several compounds of Formula (I) have substitutions at both the R1 and R2 positions.

Scheme 3 detailed below was designed to introduce a variation at the R1 and R2

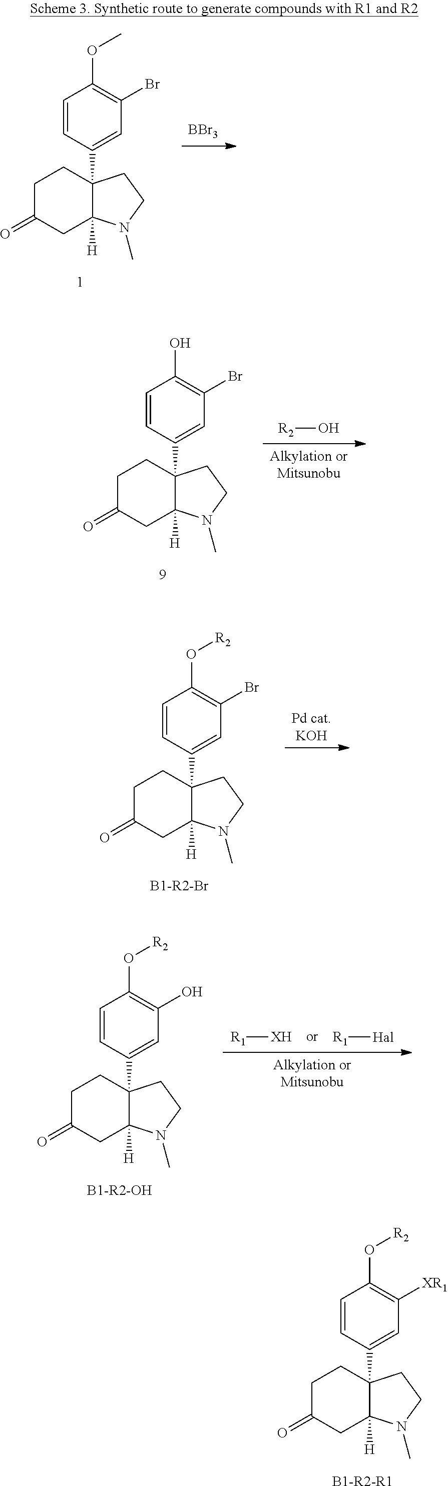

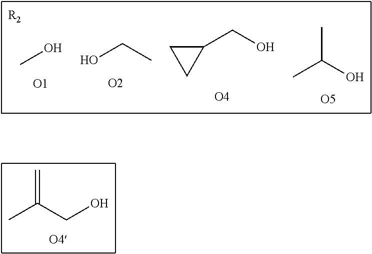

In detail, starting with the bromine intermediate prepared in Example 1, the first step was to demethylate the methoxy group with BBr₃. This reaction gave yields of 80-95% with minimal purification.

To avoid issues with purification, alkylation was chosen as a route to add R2 halides, instead of Mitsunobu. These reactions always worked to produce yields that were consistently >90% and did not require purification. Scheme 3 depicts the groups that were used for R2.

The only alkylation that was problematic for incorporating R2 was O4, the cyclopropane methyl. Under alkylation conditions this partially converted into isobutenyl (O4′).

This was partially resolved by lowering the temperature to room temperature. This resulted in a minimal formation in O4′ (˜10%) however the reaction took over 48 hours to reach completion.

Buchwald coupling conditions were tried with B1-O5-Br and ethanol. This reaction only gave trace product, which showed that Buchwald couplings were not facile with bulky R2 groups. Instead, the Pd catalyzed formation of phenols from bromides was developed. While this was an additional step, it was higher yielding, and resulted in a wider range of R1 groups that could be generated.

The only exception was for any sulfide products, which would need to be synthesized via the B1-R2-Br bromides. The palladium (Pd.) catalyzed phenol formation worked less well with bulky R2 groups, but this could often be compensated for by an increase in catalyst loading, as well as a slight raise in temperature.

For the final step of the synthesis either Mitsunobu or alkylation was used, depending on the R1 group. Scheme 4 depicts all the R1 groups and how they were coupled in the final step.

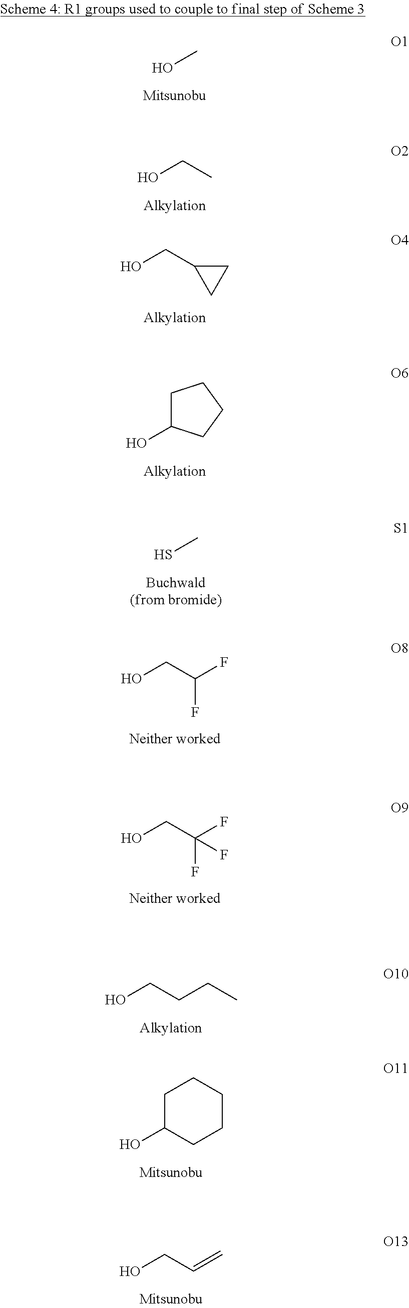

Table 2 below details the procedure used to prepare the compound and the resulting mass, yield and purity. Where relevant the compound identity number from Table 1 is detailed also.

TABLE 2
Procedures used to prepare compounds
of Formula (I) of the invention

| | | | | | Compound |
|---|---|---|---|---|---|
| | | Mass | Yield | Purity by | ID number |
| Compound | Procedure | (mg) | (%) | LC (%) | (Table 1) |
| B1-O1-O4 | A | 21 | 36 | 96.5 | 1 |
| B1-O1-O10 | A | 29 | 49 | 94.4 | 2 |
| B1-O1-S2 | B | 13 | 23 | 90.8 | 3 |
| B1-O1-O9 | A | 4 | 6 | 95.1 | 4 |
| B1-O1-O7 | A | 6 | 11 | 99.4 | 5 |
| B1-O1-O2 | A | 9 | 17 | 97.3 | 6 |
| | C | 115 | 70 | 94.8 | 6 |
| B1-O1-O8 | A | 7 | 11 | 96.4 | 7 |
| B1-O1-O6 | C | 28 | 61 | 99.6 | 11 |
| | C | 135 | 72 | 92.6 | 11 |
| B1-O1-O12 | A | 2 | 3 | 90.1 | 12 |
| B1-O1-O13 | D | 13 | 38 | 95.0 | 13 |
| | D | 168 | 61 | 97.3 | 13 |
| B1-O1-O11 | D | 30 | 57 | 98.8 | 14 |
| B1-O1-O14 | D | 1 | 2 | 91.6 | 15 |
| B1-O1-S1 | B | 7 | 12 | 61.4 | 17 |
| B1-O5-O1 | D | 14 | 54 | 79.3 | 19 |
| B1-O2-O10 | C | 16 | 70 | 99.0 | 20 |
| B1-O2-O2 | C | 14 | 67 | 97.0 | 21 |
| | C | 100 | 61 | 93.1 | 21 |
| B1-O5-O4 | C | 14 | 59 | 97.0 | 22 |
| B1-O2-O11 | D | 15 | 47 | 91.8 | 23 |
| B1-O2-O4 | C | 8 | 35 | 98.1 | 24 |
| | C | 76 | 43 | 98.3 | 24 |
| | C | 28 | 39 | 97.4 | 24 |
| B1-O5-S1 | B | 13 | 28 | 94.7 | 25 |
| B1-O5-O11 | D | 12 | 38 | 90.2 | 26 |
| B1-O2-O6 | C | 12 | 51 | 99.1 | 27 |
| | C | 116 | 63 | 99.1 | 27 |
| B1-O5-O6 | C | 12 | 49 | 99.0 | 28 |
| B1-O5-O2 | C | 14 | 64 | 97.8 | 29 |
| B1-O5-O10 | C | 17 | 72 | 99.0 | 30 |
| B1-O2-O1 | D | 13 | 51 | 96.9 | 31 |
| B1-O4-O1 | D | 13 | 37 | 95.1 | 34 |
| B1-O4-O2 | C | 14 | 52 | 93.3 | 35 |
| B1-O4-O4 | C | 10 | 34 | 98.1 | 36 |
| B1-O4-O10 | C | 12 | 41 | 99.3 | 37 |
| B1-O4-O13 | D | 12 | 31 | 91.4 | 38 |
| B1-O4-O6 | C | 11 | 36 | 98.4 | 39 |

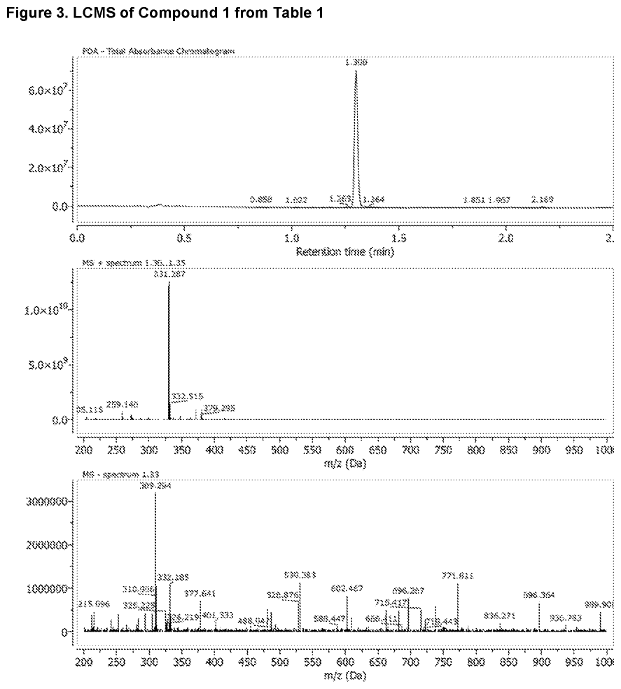

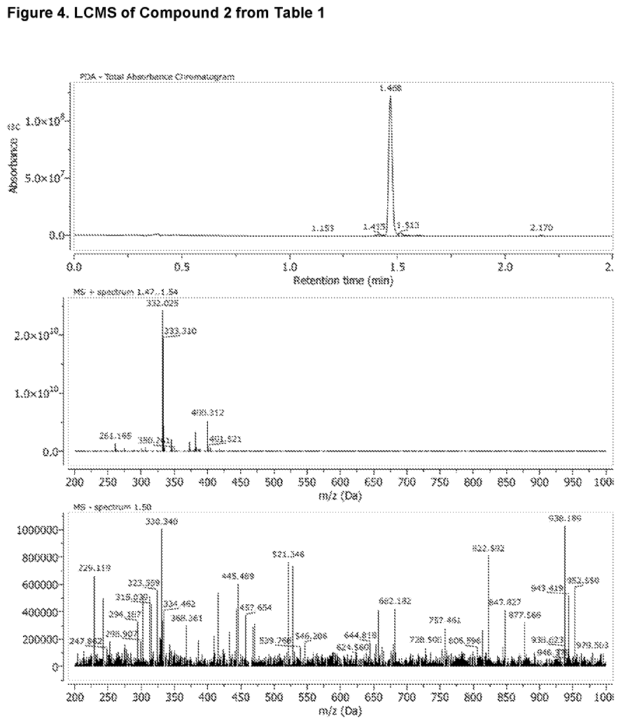

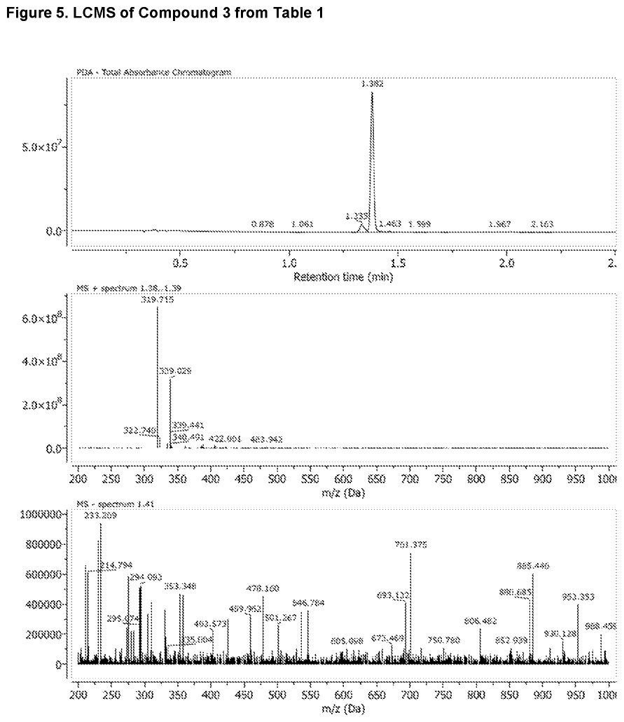

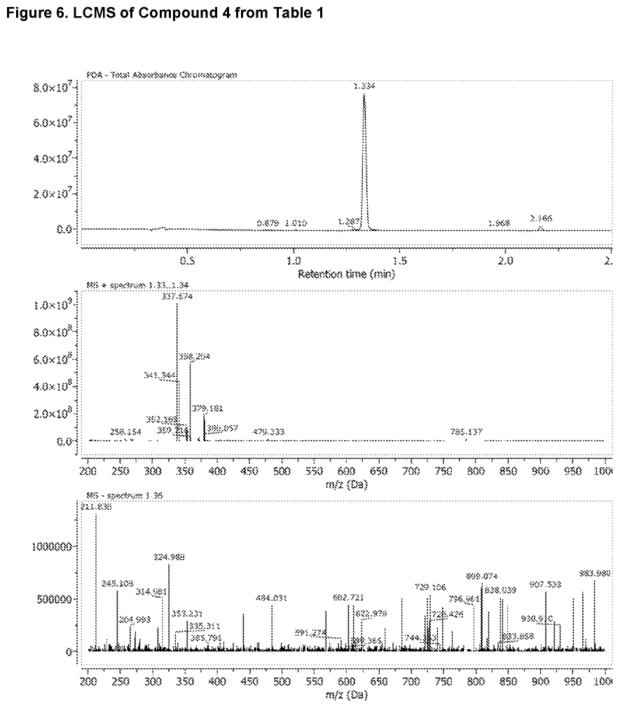

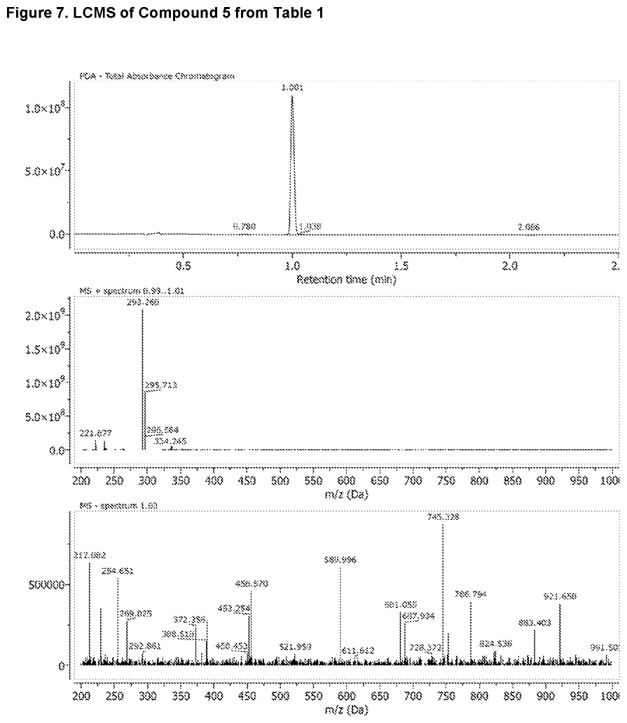

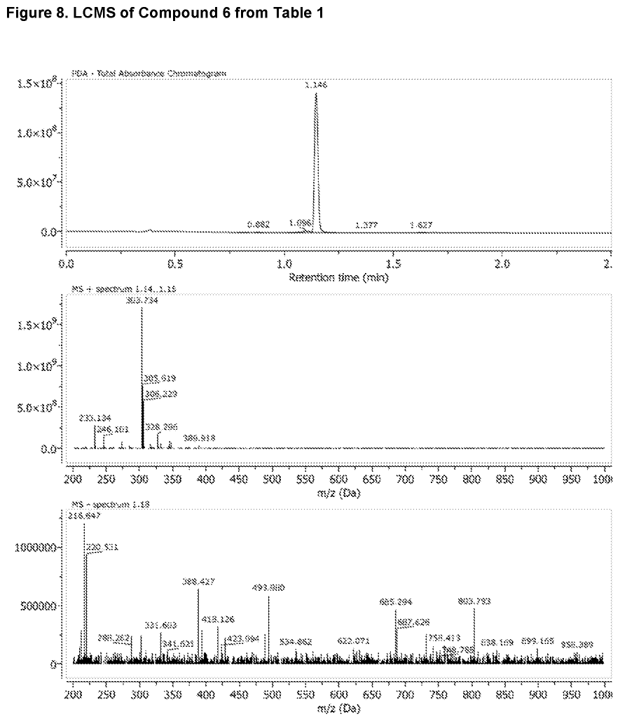

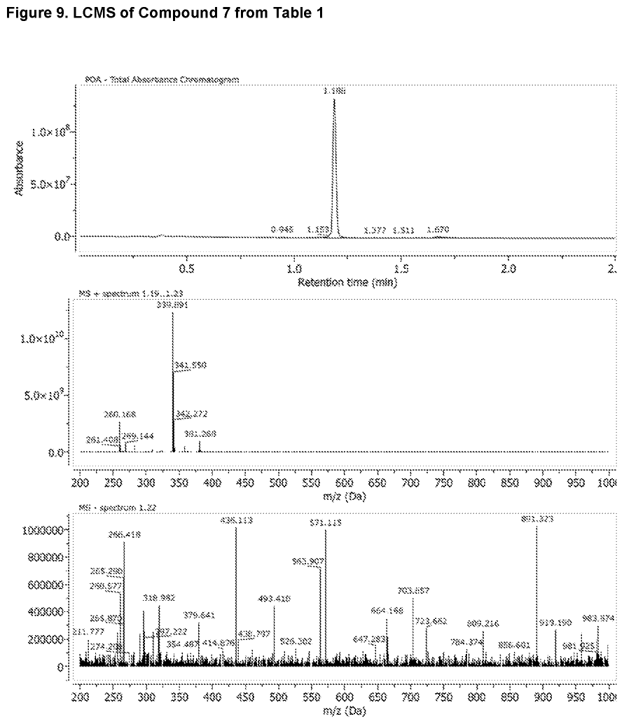

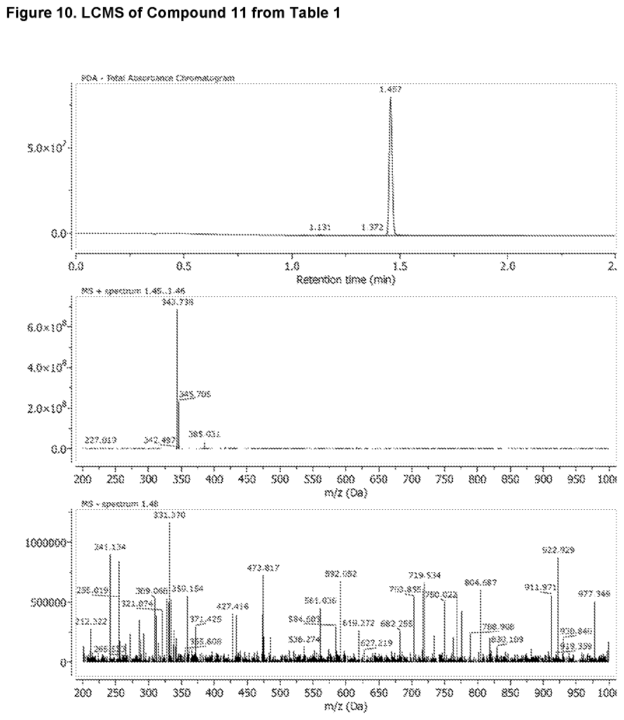

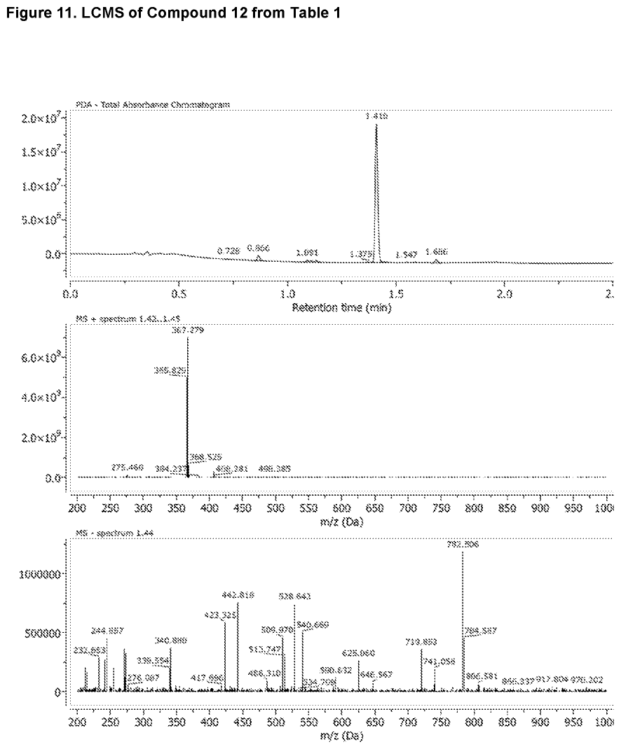

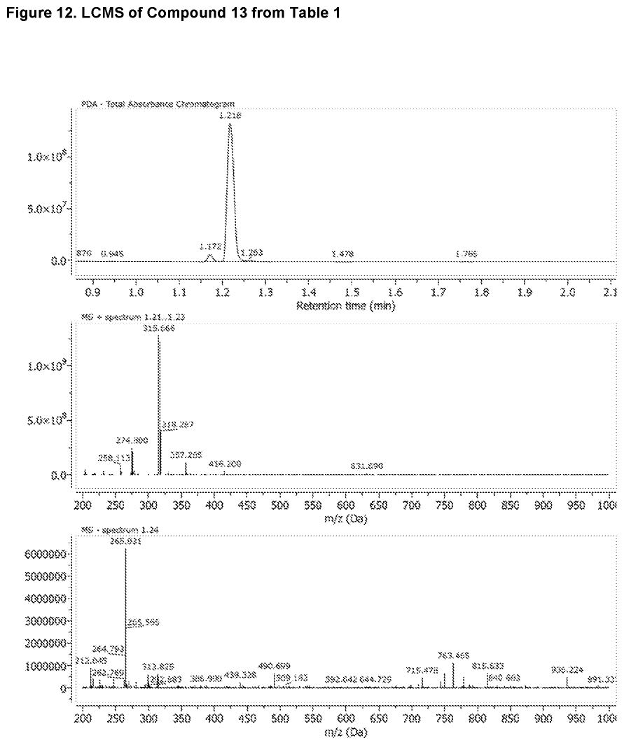

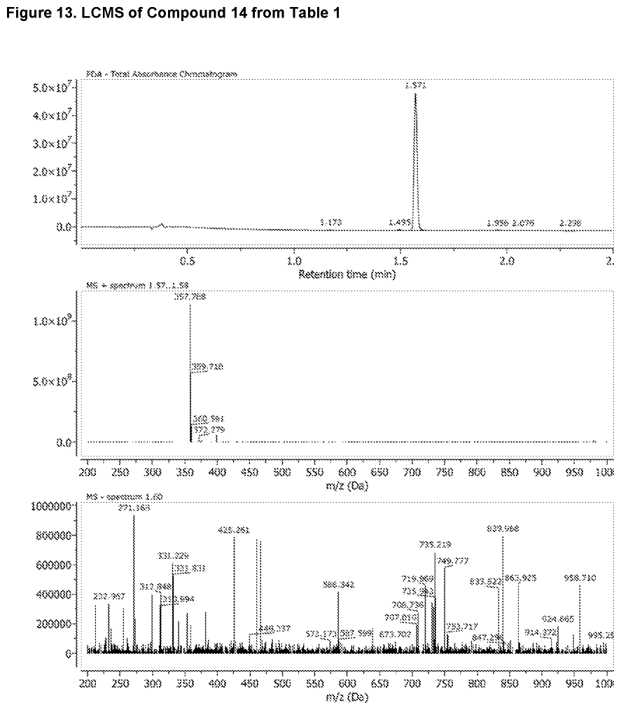

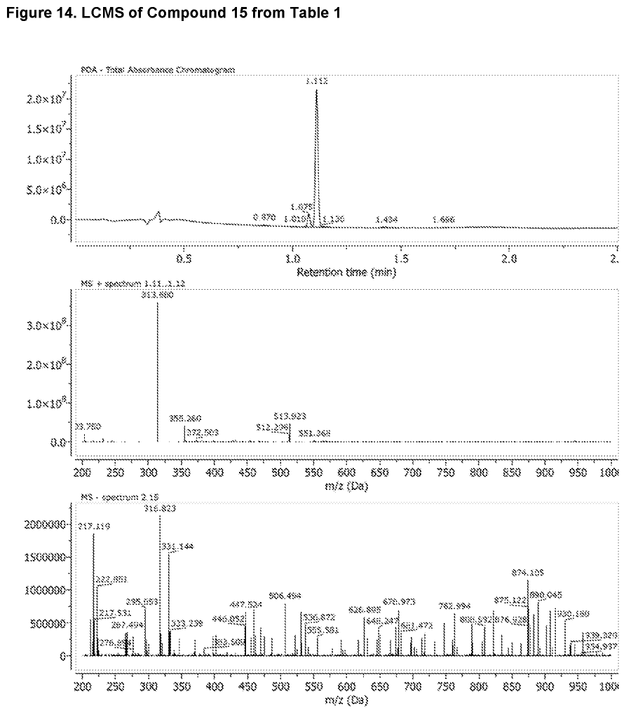

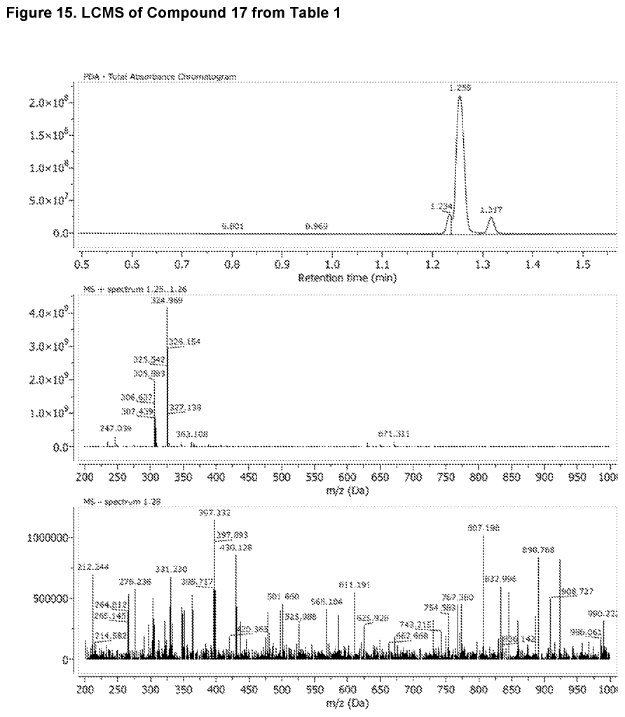

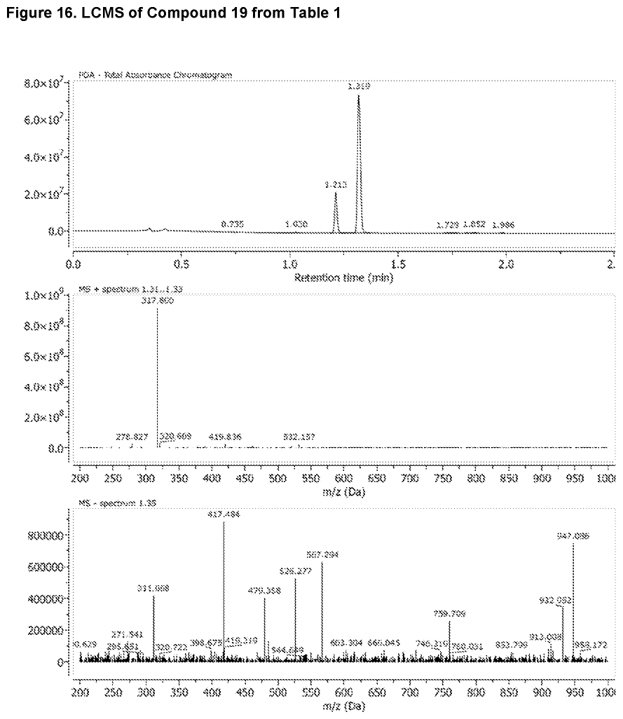

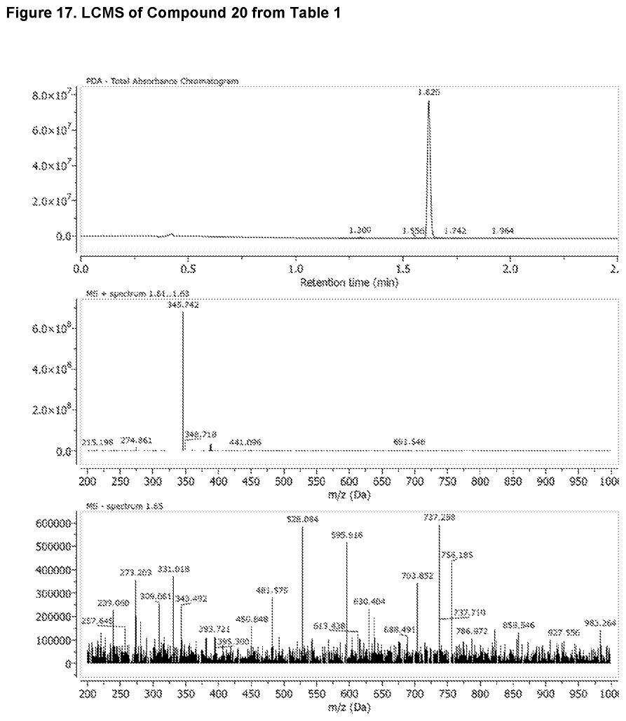

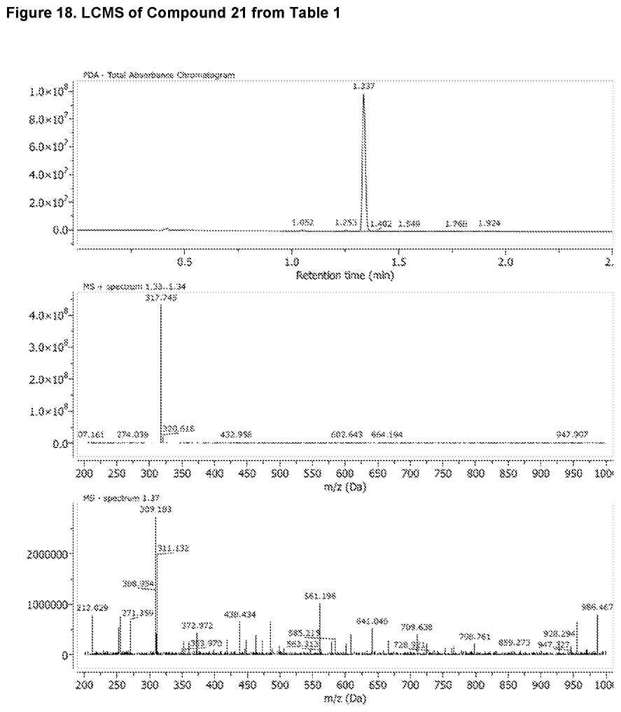

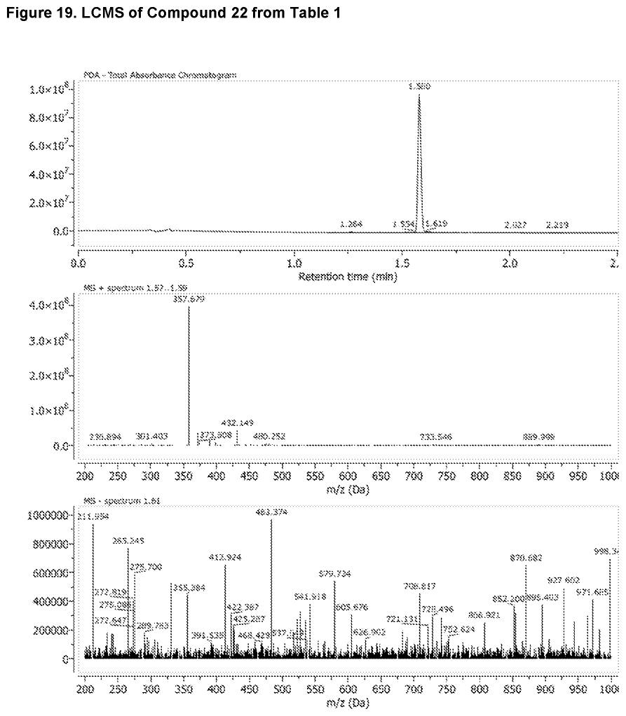

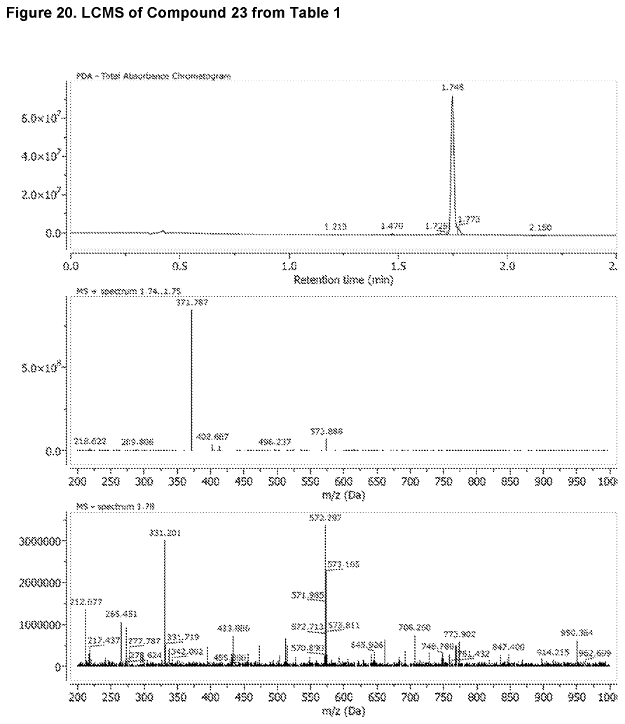

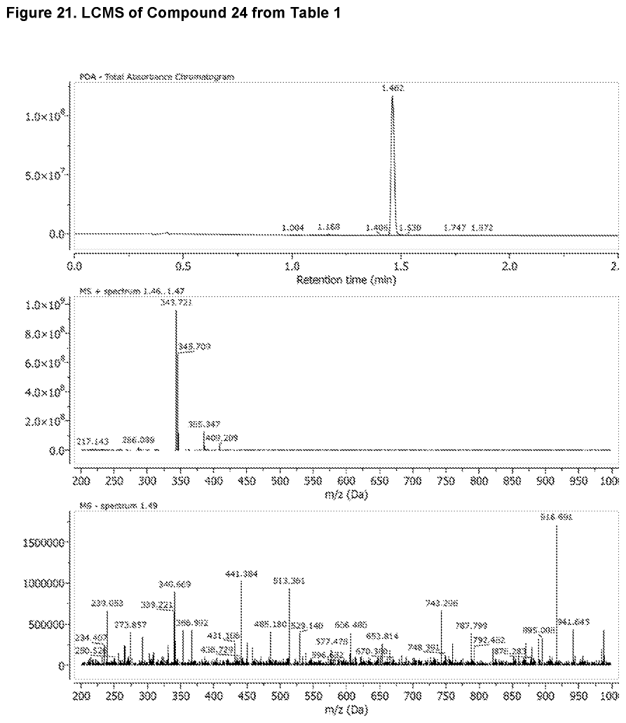

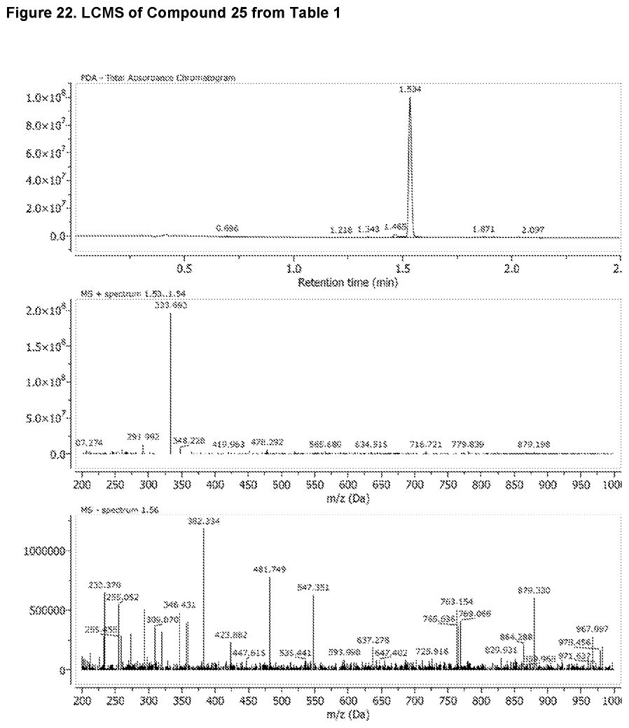

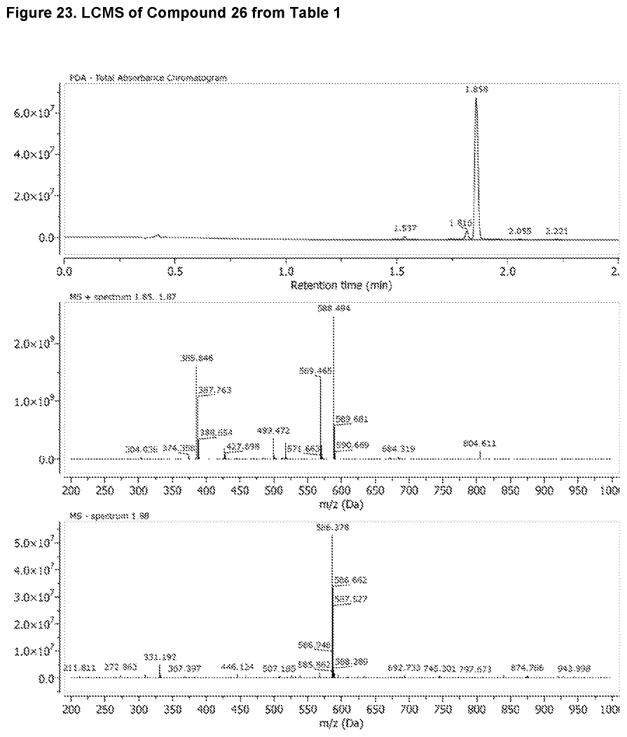

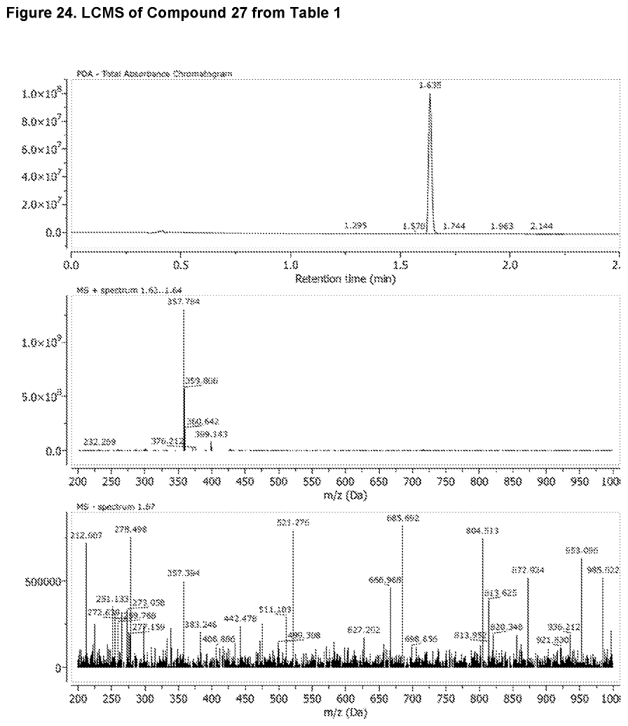

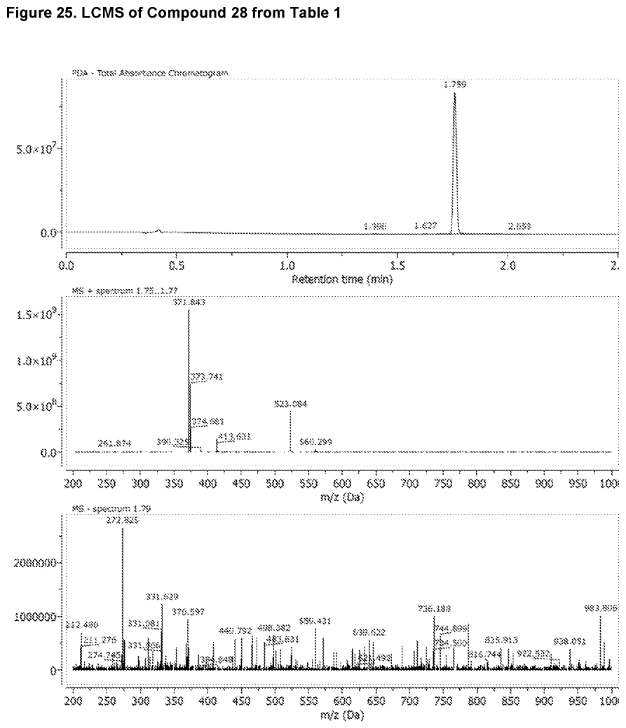

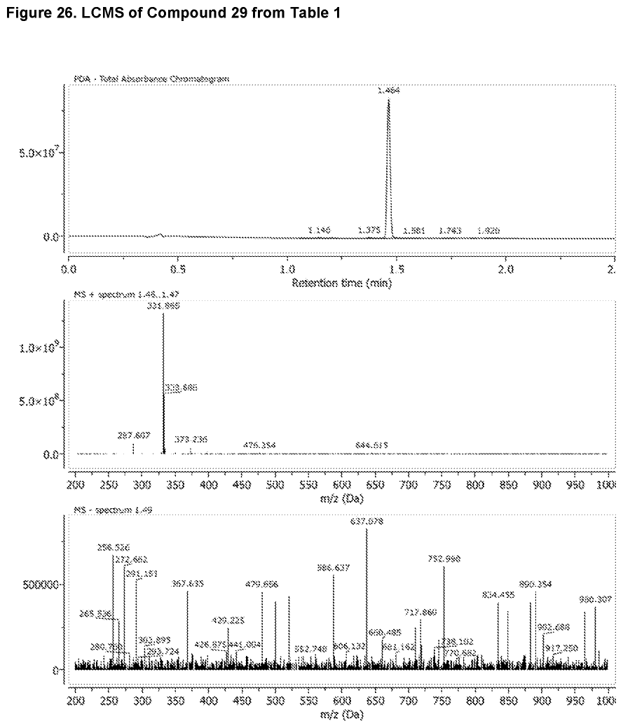

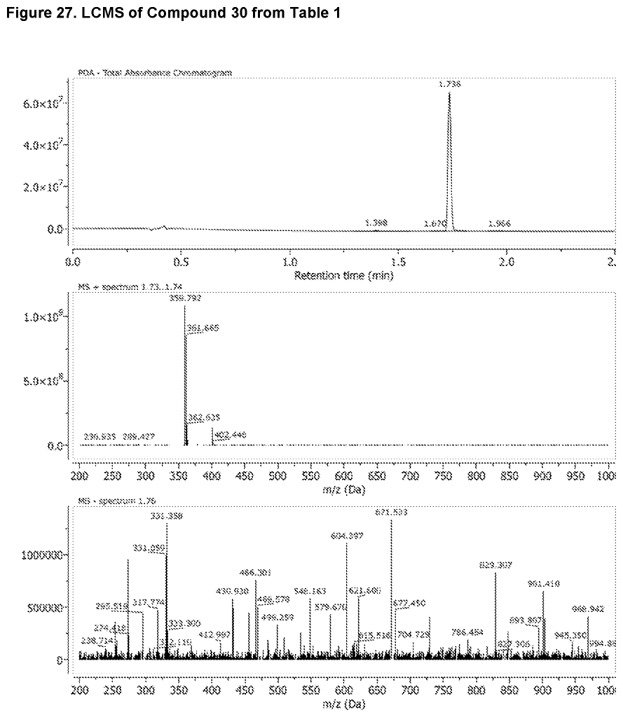

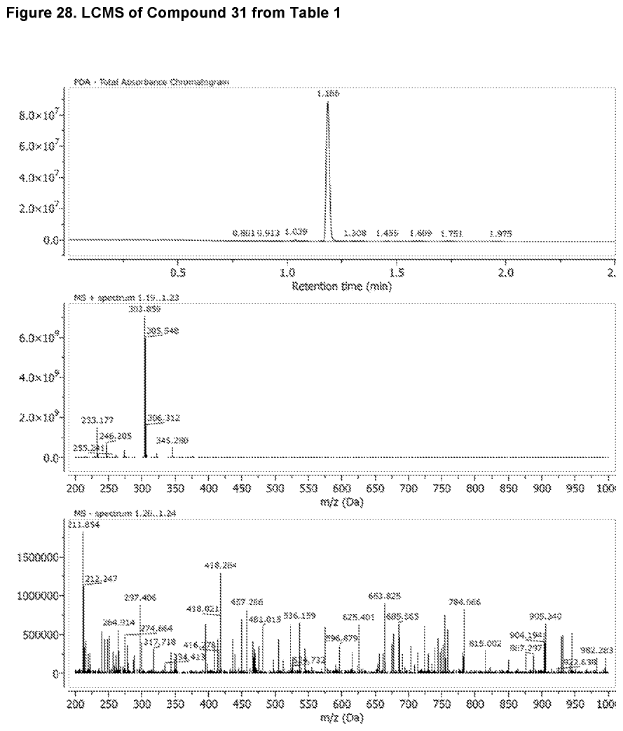

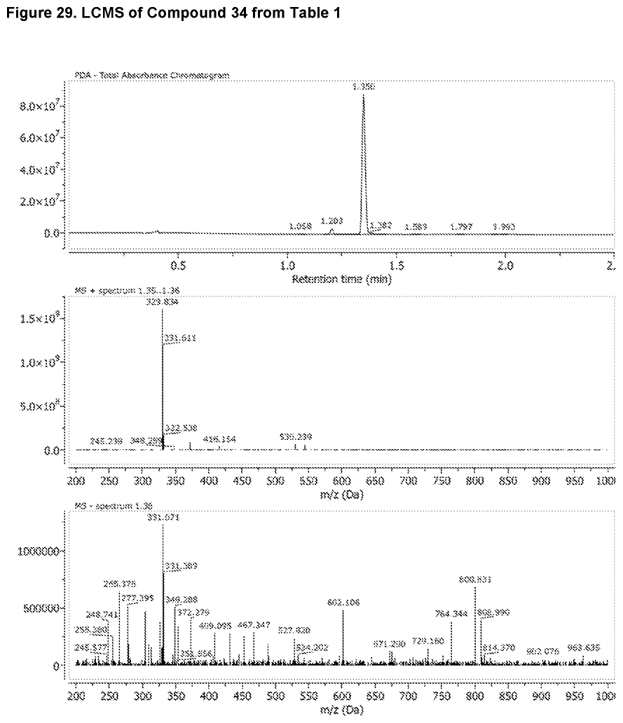

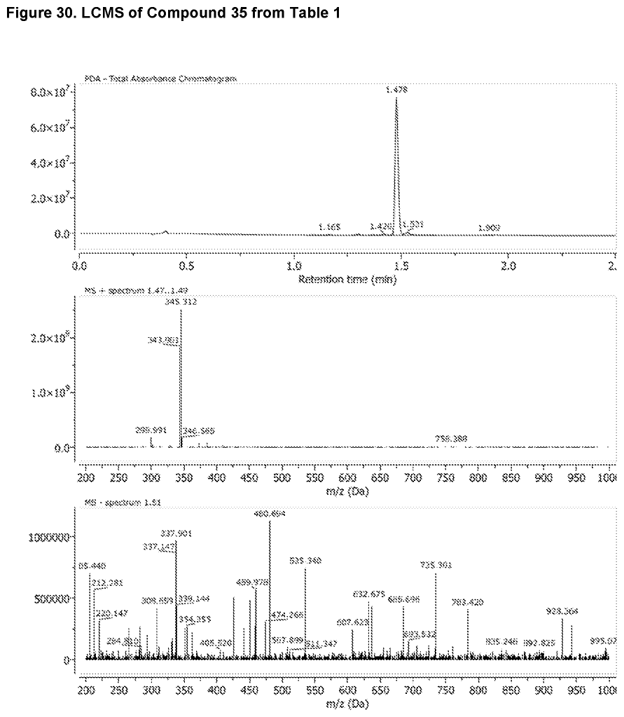

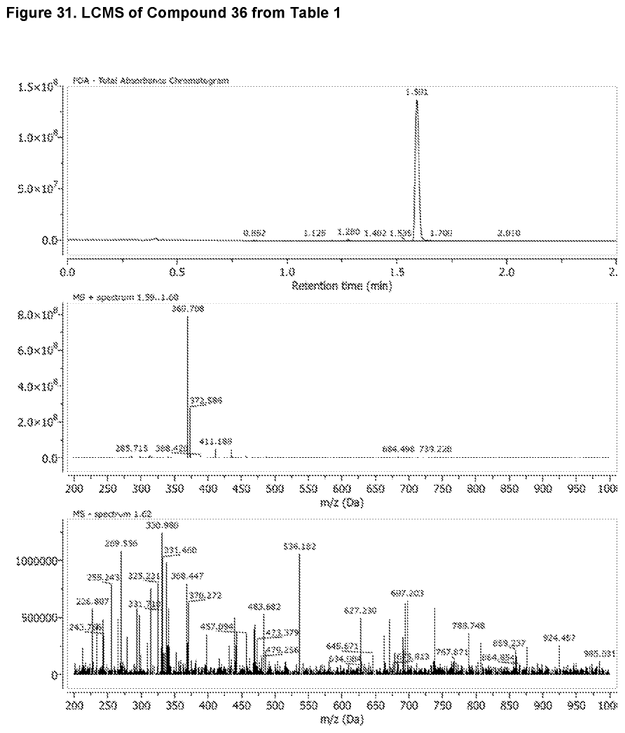

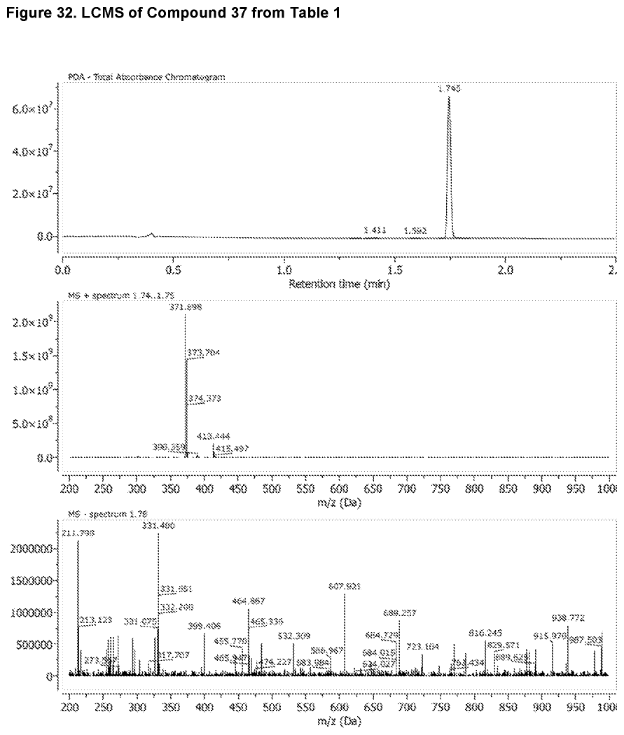

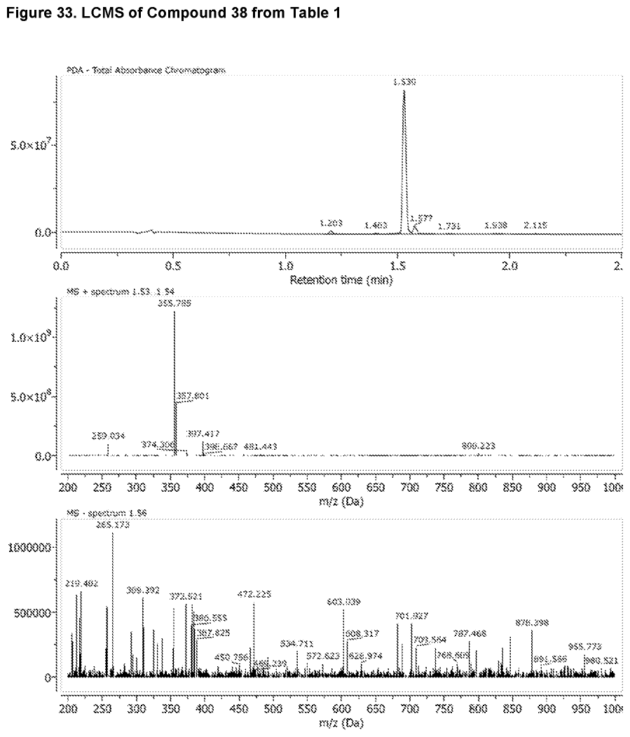

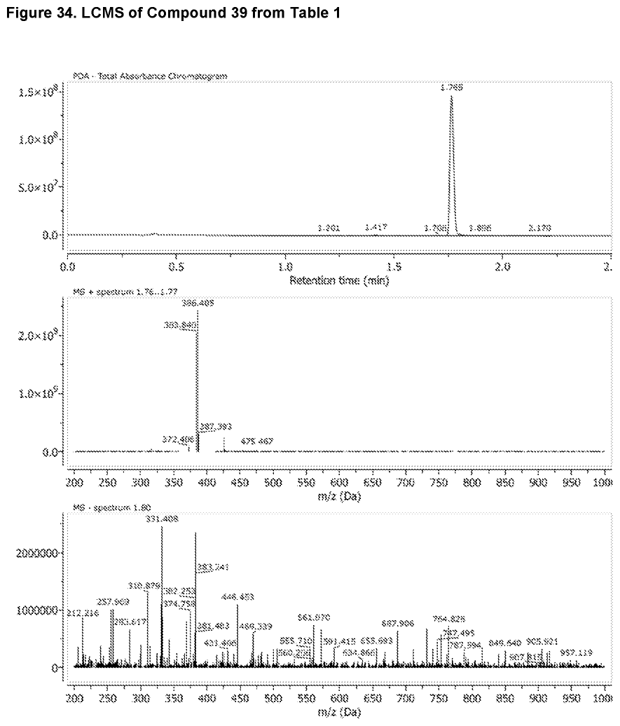

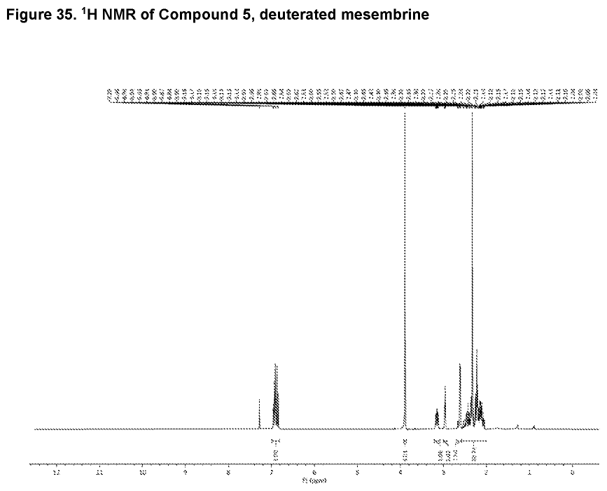

The compounds listed in Table 2 above were tested by LCMS, FIGS. 3 to 35 detail the spectra for these compounds.

Compounds 40-43 of Table 1 were prepared following the synthetic route Scheme 5 below.

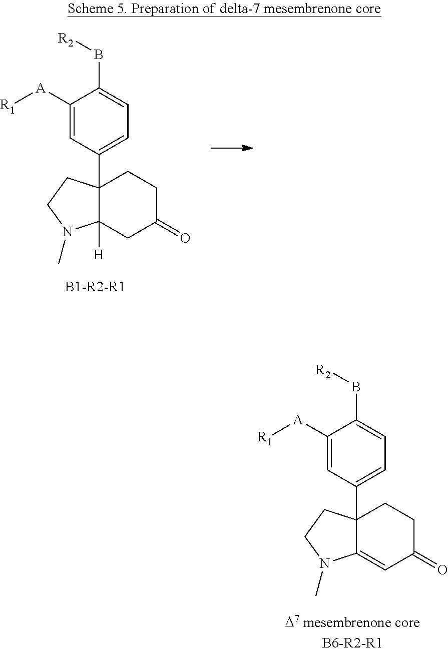

A freshly prepared solution of LDA was added to a solution of the required mesembrenone compound (e.g., Compound 11) in THF (2 ml) at 0° C. The reaction mixture was stirred for 10 minutes and TMS-Cl was added.

After stirring for 1 hour at 0° C. a second addition of LDA was added followed by the addition of N-(1,1-dimethylethyl)benzenesulfinimidoyl chloride. The reaction was left to slowly warm to room temperature and stirring for 16 hours.

The reaction was quenched by the addition of HCl 1N and stirred for another 2 hours. The mixture was quenched with a solution of sat. NaHCO₃ was added until pH ˜7 and the aqueous phase was extracted with DCM (3×5 mL).

The combined organic layers were dried over Na₂SO₄, filtered and concentrated under reduced pressure to give a brown oil which was further purified by Prep chromatography.

To prepare Compound 44 of Table 1 a mesembrenone analogue needed to be prepared. Scheme 6 below details the steps involved.

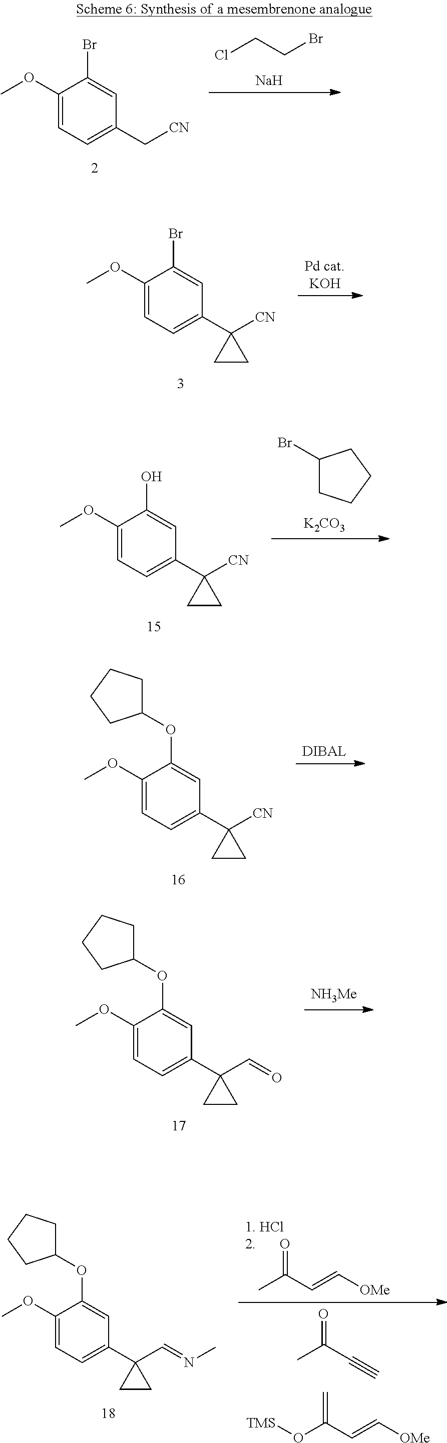

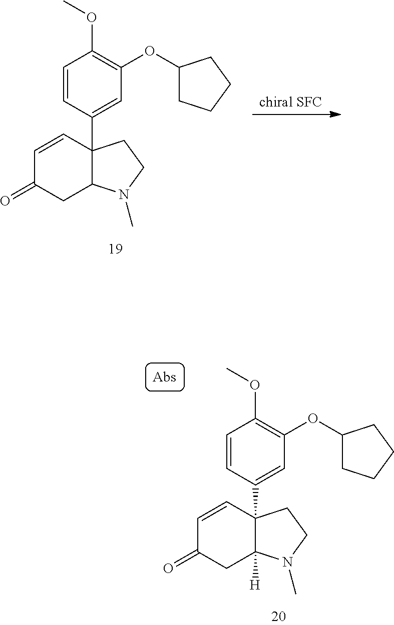 [SMILES: COc1ccc(C23C=CC(=O)CC2N(C)CC3)cc1OC1CCCC1 ; "chiral SFC" ; COc1ccc([C@@]23C=CC(=O)C[C@@H]2N(C)CC3)cc1OC1CCCC1]

Acetonitrile 2 was converted to cyclopropane 3, which was then converted to phenol 15 using Pd₂(dba)₃, tBuBrettphos and KOH in water/dioxane with 81% yield. This was then alkylated with cyclopentylbromide to form 16 with quantitative yields.

Compound 16 was reduced to aldehyde 17 with DIBAL with an 85% yield. Aldehyde 17 could be converted to imine 18 with methylamine, also with quantitative yields.

Cyclopropane 18 was converted to the unisolated dihydropyrrole, then it was combined with methyl 3-methoxyacrylate and Na₂SO₄ in MeCN and refluxed.

To prepare diethyl mesembrenone, (Compound 45 of Table 1), the following Scheme 7 was utilised.

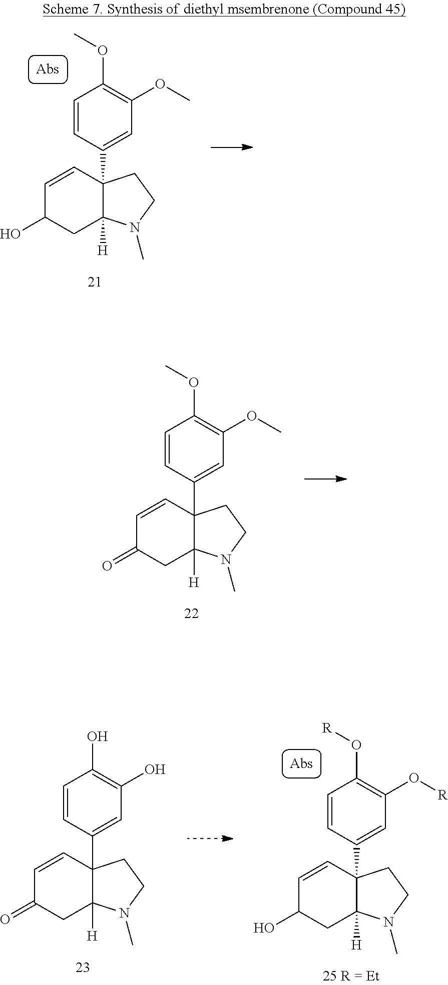

Mesembrenol could be converted to mesembrenone 22 using Dess-Martin periodinane, with a 92% yield.

Mesembrenone 22 was converted to the di-desmethyl-Mesembrenone 23 using BBr₃,

Di-desmethyl-Mesembrenone 23 was combined with Etl and K₂CO₃ to form 25.

### Example 3: Preparation of Deuterated Mesembrine

Preparation of deuterated mesembrine, compound 5 from Table 1, was undertaken using the following method and as detailed in Scheme 8 below.

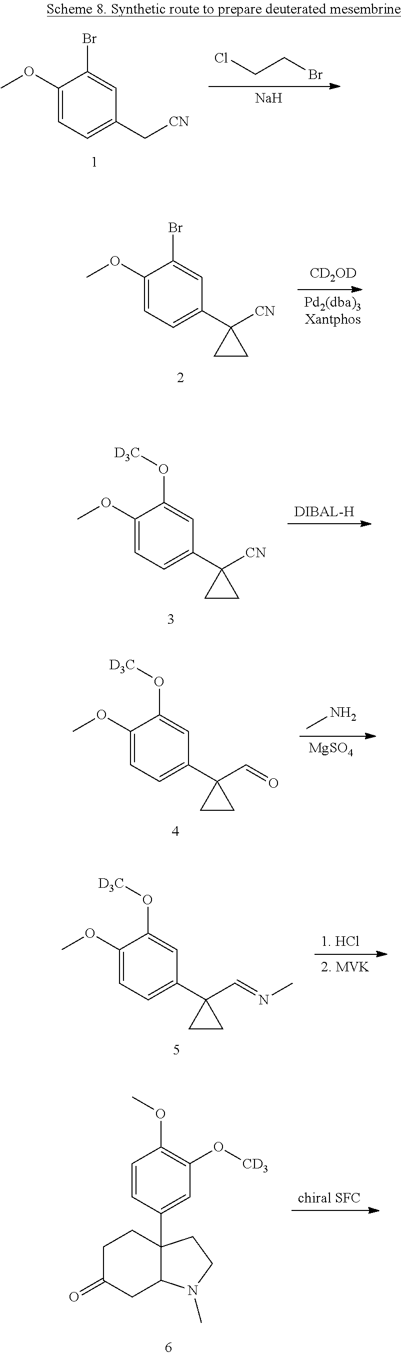

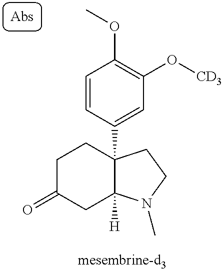 [SMILES: [2H]C([2H])([2H])Oc1cc([C@@]23CCC(=O)C[C@@H]2N(C)CC3)ccc1OC]

The cyclopropane formation was carried out on 10 gram scale with 1-bromo-2-chlorethane and NaH as base and afforded the desired compound 2 in 81% yield.

Substitution of the bromide under Buchwald conditions in the presence of CD30D gave the deuterated compound 3 in 96% after column chromatography.

DIBAL-H reduction of the nitrile at −4° C. is fast. Work-up needs stirring of the reaction mixture with 1N HCl for at least 30 min to break all the aluminum salts. To completely remove the salts the aldehyde is flushed of silica affording pure aldehyde in 82% yield.

The imine formation is rather slow and needs several days to run to completion but after filtration of the MgSO₄ the imine is isolated quantitively. The imine is immediately used in the next step.

Treatment of the imine with HCl in diethyl ether gave the ring-expansion and subsequently the intermediate is treated with methyl vinyl ketone to afford the racemic mesembrine in 37% yield after column chromatography.

SFC separation of the enantiomers afforded 375+335 mg of the desired enantiomer with an e.e. of 97.1% and a purity of 96%. For the SFC separation two batches of racemic material were used (1.4 g and 1.0 g).

### Example 4: Preparation of the Compounds of Formula (II)

Preparation of the compounds of Formula II were prepared as described in Schemes 9 to 15 below. The target number used in the schemes corresponds to the compound number from Table 1 as shown below in Table 3:

TABLE 3
Identification of Target number from schemes

| Compound number | Target number in |
|---|---|
| from Table 1 | schemes |
| 46 | 1 |
| 47 | 2 |
| 48 | 3 |
| 49 | 4 |
| 50 | 5 |
| 51 | 6 |
| 52 | 7 |
| 53 | 8 |
| 54 | 9 |
| 55 | 10 |
| 56 | 11 |
| 57 | 12 |
| 58 | 13 |
| 59 | 14 |
| 60 | 15 |
| 61 | 16 |

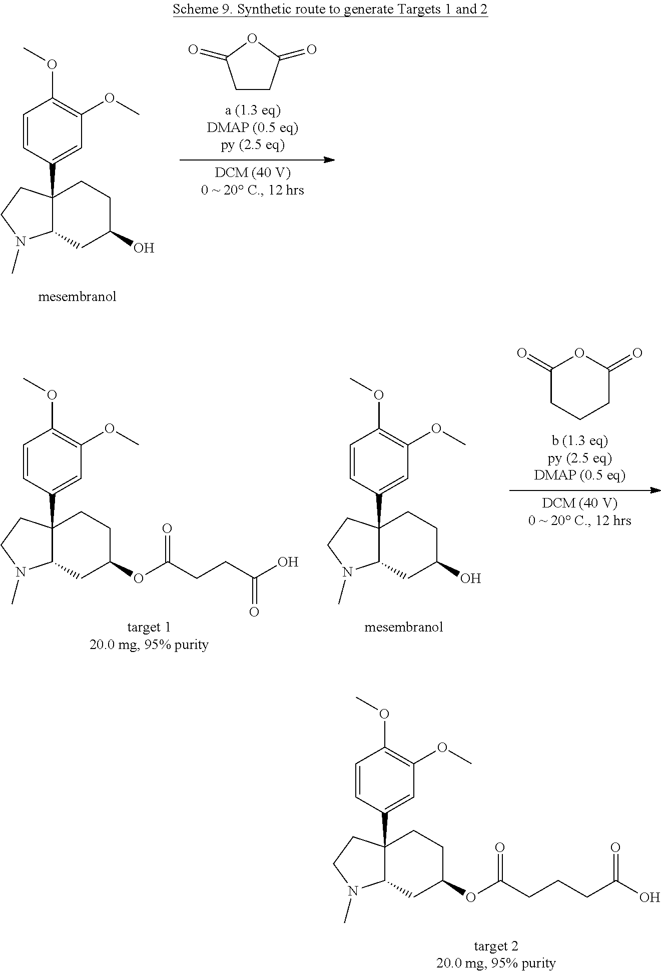

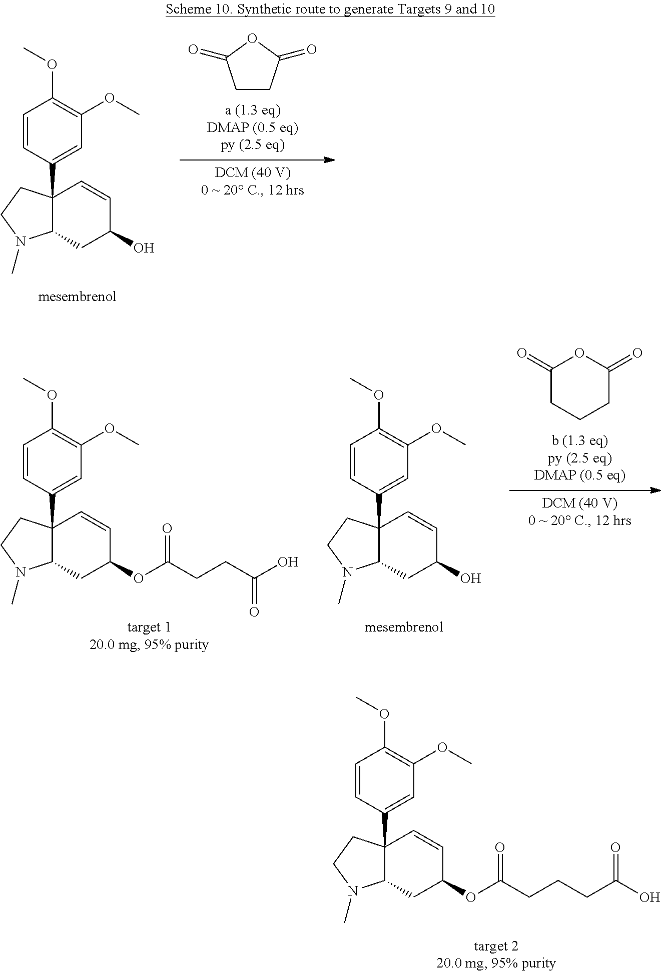

DMAP (0.5 eq) and Py (2.5 eq) was added to a solution of compound a (1.3 eq) or compound b (1.3 eq) in DCM (20 V) at 0° C. A solution of mesembranol (1.0 eq) or mesembrenol (1.0 eq) in DCM (20 V) was added to the reaction at 0° C. The reaction was stirred at 20° C. for 12 hrs. LCMS showed the starting material was consumed completely.

The reaction was poured into ice water (80 V), extracted with DCM (50 V). The organic layer was concentrated under reduce pressure to obtain the crude product. Then purified by prep-HPLC (column: Waters Xbridge BEH C18 100×30 mm×10 μm; mobile phase: [water (NH₄HCO₃)— ACN]; B %: 5%-30%, 8 mins), then lyophilized to obtain Targets 1, 2, 9 and 10 as light-yellow solid.

Scheme 11. Synthetic route to generate Targets 3 and 4

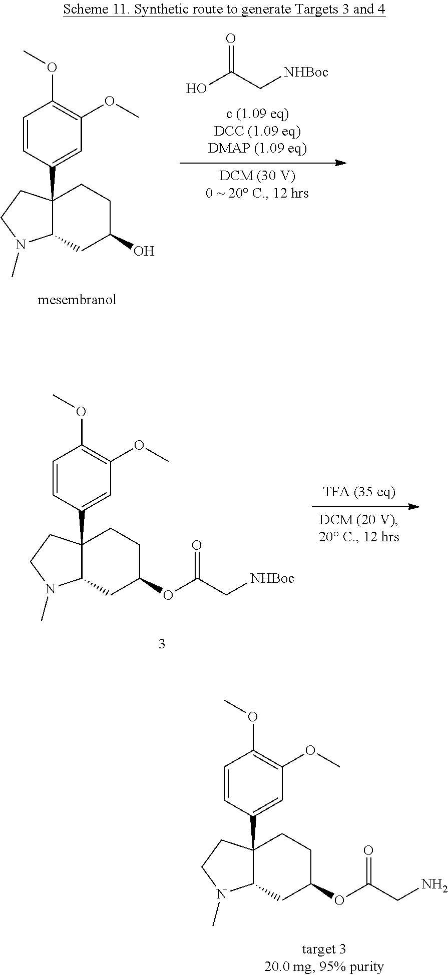

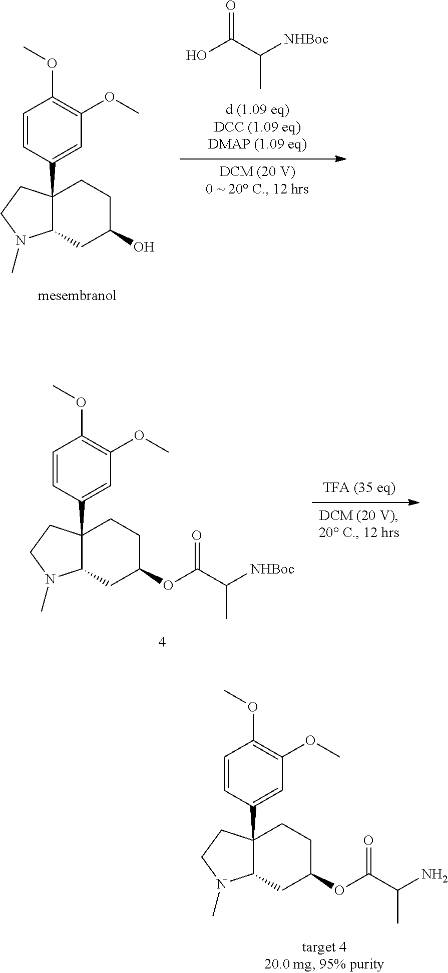

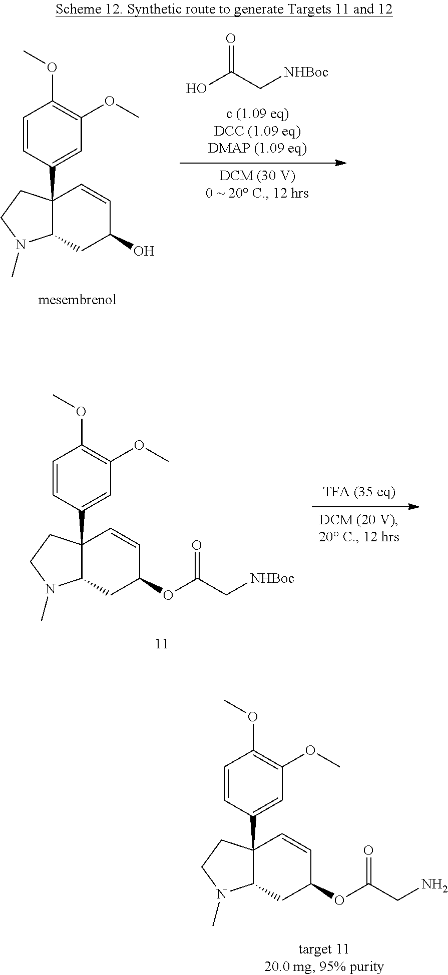

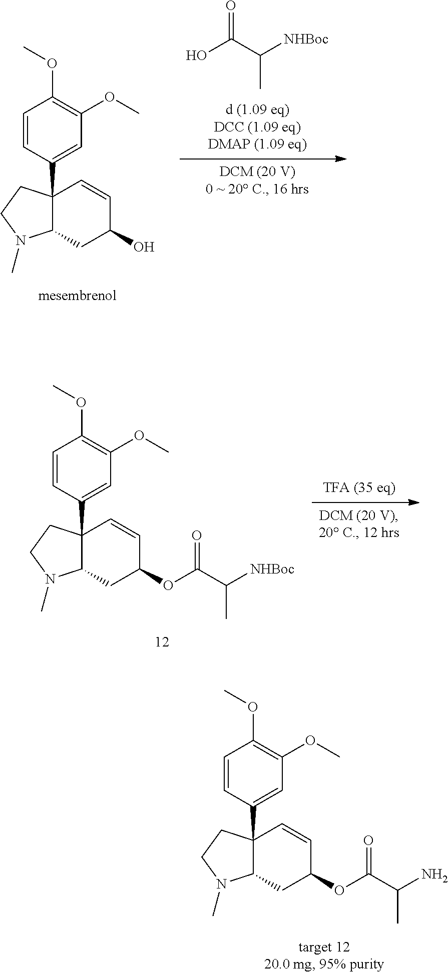

To a solution of mesembranol (1.0 eq) or mesembrenol (1.0 eq) in DCM (30 V) was added compound c (1.09 eq) or compound d (1.09 eq) at 0° C. Then DMAP (1.09 eq), DCC (1.09 eq) was added to the reaction. The reaction was stirred at 20° C. for 12 hrs. LCMS showed the reaction was worked well. The reaction was quenched by water (45 V), then extracted with DCM (45 V). The organic layer was concentrated under reduced pressure to obtain the crude product. Compounds 3, 4, 11 and 12 (crude) was obtained as yellow oil and used to next step without further purification.

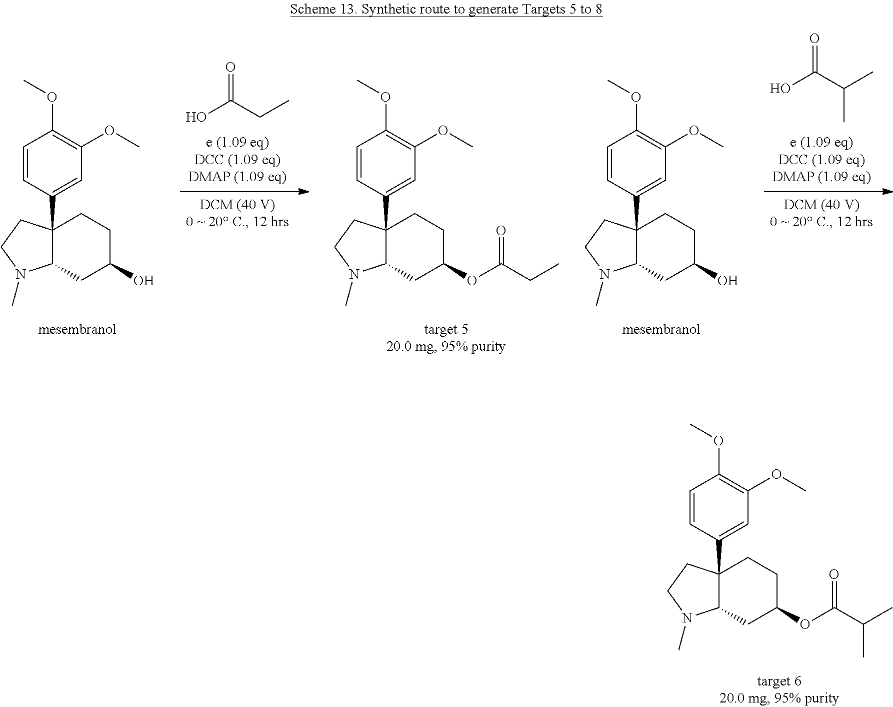

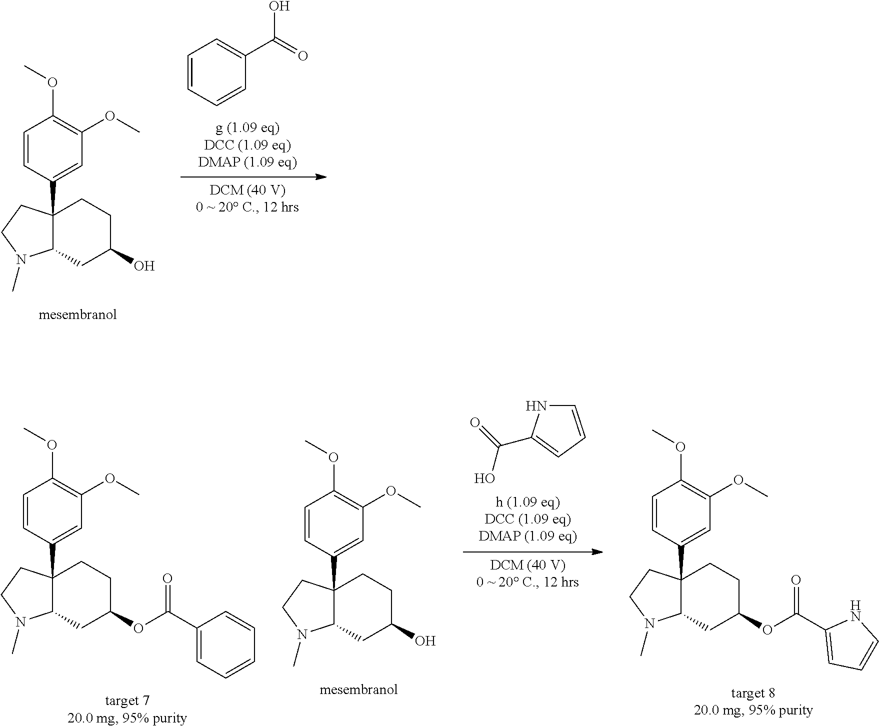

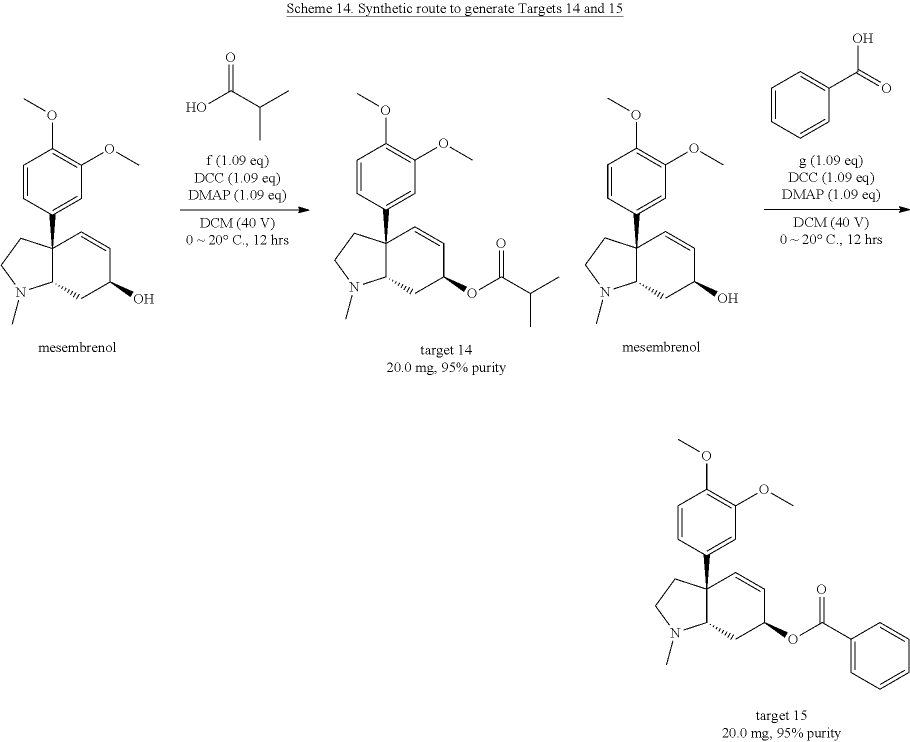

DCC (1.09 eq) and DMAP (1.09 eq) was added to a solution of compound e (1.09 PGP-1I1,C₃ eq) or compound f (1.09 eq) or compound g (1.09 eq) or compound h (1.09 eq) in DCM (20 V) at 0° C. A solution of mesembranol (1.0 eq) or mesembrenol (1.0 eq) in DCM (20 V) was added to the reaction at 0° C. The reaction was stirred at 20° C. for 12 hrs. LCMS showed the reaction was worked well. The reaction was quenched by water (50 V), extracted with DCM (50 V). The organic layer was concentrated under reduced pressure to obtain the crude product. Then purified by prep-HPLC (column: Waters Xbridge BEH C18 100×30 mm×10 um; mobile phase: [water (NH4HCO3)— ACN]; B %: 30%-60%, 8 mins), then lyophilized to obtain Targets 5, 6, 7, 8, 14 and 15 as yellow oil.

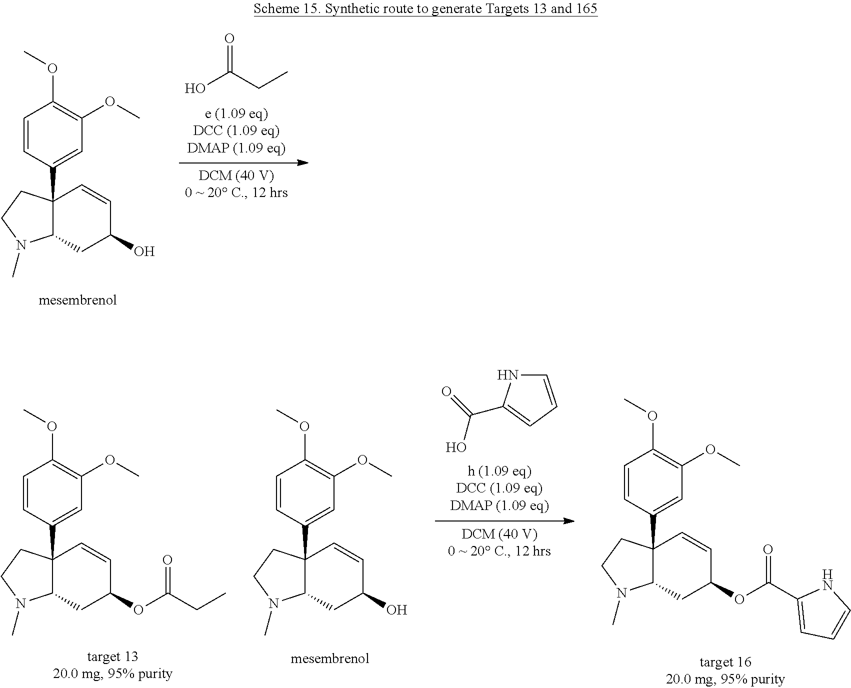

DCC (1.09 eq) and DMAP (1.09 eq) was added to a solution of compound e (1.09 eq) or compound h (1.09 eq) in DCM (20 V) at 0° C. A solution of mesembrenol (1.0 eq) in DCM (20 V) was added to the reaction at 0° C. The reaction was stirred at 20° C. for 12 hrs. LCMS showed the starting material was consumed completely. The reaction was poured into ice water (40 V), extracted with DCM (30 V). The organic layer was concentrated under reduced pressure to obtain the crude product. Then purified by prep-HPLC (column: Phenomenex C18 80×40 mm×3 um; mobile phase: [water (NH4HCO3)— ACN]; B %: 30%-60%, 8 mins). Then lyophilized to obtain Targets 13 and 16 as colorless oil. LCMS showed the product was remained ˜12% by-product from DCC, so, re-purified by triturate with ACN (0.4 mL) at 0° C. for 30 mins, filtered and the filtrate was concentrated under reduce pressure to obtain the product.

Table 4 provides details of the ¹H NMR data produced for the 16 different target compounds in addition to the amount produced and the yield.

TABLE 4
Properties of compounds produced using Schemes 9 to 15

| Compound | | Amount | |
|---|---|---|---|
| ID | ¹H NMR | (mg) | Yield |
| Target 1 | ET56634-24-P1A (400 MHz, CDCl₃) | 20.37 | 20.3% |
| | δ 6.82-6.91 (m, 3H), 5.10-5.15 (m, 1H), 3.90 (s, 3H), 3.88 (s, | | |
| | 3H), 3.50 (br. s, 1H), 3.24 (br. s, 1H), 2.46-2.57 (m, 8H), 2.01- | | |
| | 2.16 (m, 5H), 1.78-1.85 (m, 2H), 1.37-1.42 (m, 1H). | | |
| Target 2 | ET56634-25-P1A (400 MHz, CDCl₃) | 19.80 | 20.1% |
| | δ 6.82-6.90 (m, 3H), 5.09-5.16 (m, 1H), 3.90 (s, 3H), 3.88 (s, | | |
| | 3H), 3.47 (brs, 1H), 3.11 (br. s, 1H), 2.48 (s, 1H), 2.32-2.34 (m, | | |
| | 4H), 2.30-2.31 (m, 1H), 1.76-2.07 (m, 8H), 1.29-1.34 (m, 1H). | | |
| Target 3 | ET56634-37-P1A2 (400 MHz, MeOD) | 19.33 | 16.3% |
| | δ 6.94-7.03 (m, 3H), 5.20-5.34 (m, 1H), 4.20 (brs, 1H), 3.77-3.96 | | |
| | (m, 10H), 3.32-3.37 (m, 1H), 3.06 (s, 3H), 2.54 (d, J = 15.2 Hz, | | |
| | 1H), 2.41 (d, J = 15.2 Hz, 1 H), 2.26-2.33 (m, 2H), 1.96-2.12 (m, | | |
| | 2H), 1.81-1.92 (m, 1H), 1.35-1.47 (m, 1H). | | |
| Target 4 | ET56634-38-P1B (400 MHz, MeOD) | 27.77 | 15.8% |
| | δ 6.99 (brs, 3H), 5.23 (brs, 1H), 4.02-4.09 (m, 1H), 3.74-3.98 (m, | | |
| | 9H), 3.07 (s, 3H), 2.54 (d, J = 14.2 Hz, 1 H), 2.43 (d, J = 14.2 | | |
| | Hz, 1H), 2.21-2.34 (m, 2H), 1.94-2.12 (m, 3H) 1.81-1.87 (m, 1 H) | | |
| | 1.52 (d, J = 7.2 Hz, 3H), 1.33-1.47 (m, 1H). | | |
| Target 5 | ET56634-16-P1A1 (400 MHz, CDCl₃) | 23.75 | 24.0% |
| | δ 6.80-6.97 (m, 3H), 4.99-5.13 (m, 1H), 3.89 (d, J = 7.2 Hz, 6H), | | |
| | 3.16-3.26 (m, 1H), 2.75 (s, 1H), 2.37 (s, 3H), 2.22-2.32 (m, 3H), | | |
| | 2.02-2.22 (m, 3H), 1.86-1.95 (m, 1H), 1.76-1.83 (m, 2H), 1.54- | | |
| | 1.63 (m, 1H), 1.24-1.38 (m, 1H), 1.10 (t, J = 7.6 Hz, 3H). | | |
| Target 6 | ET56634-9-P1A (400 MHz, CDCl₃) | 21.80 | 32.2% |
| | δ 6.81-6.96 (m, 3H), 5.01-5.12 (m, 1H), 3.89 (d, J = 7.0 Hz, 6H), | | |
| | 3.19-3.24 (m, 1H), 2.75 (s, 1H), 2.41-2.48 (m, 1H), 2.37 (s, 3H), | | |
| | 2.25-2.32 (m, 1H), 2.02-2.21 (m, 3H), 1.85-1.95 (m, 1H), 1.74- | | |
| | 1.84 (m, 2H), 1.53-1.62 (m, 1H), 1.26-1.35 (m, 1H), 1.13 (d, J = | | |
| | 3.2 Hz, 3H), 1.11 (d, J = 3.2 Hz, 3H). | | |
| Target 7 | ET56634-10-P1A1 (400 MHz, CDCl₃) | 22.19 | 29.2% |
| | δ 7.98-8.03 (m, 2H), 7.49-7.57 (m, 1H), 7.37-7.44 (m, 2H), 6.95- | | |
| | 6.99 (m, 1H), 6.92 (d, J = 2.0 Hz, 1H), 6.86 (d, J = 8.4 Hz, 1H), | | |
| | 5.27-5.39 (m, 1H), 3.91 (d, J = 7.6 Hz, 6H), 3.21-3.29 (m, 1H), | | |
| | 2.82 (s, 1H), 2.42 (s, 3H), 2.28-2.38 (m, 2H), 2.10-2.26 (m, 2H), | | |
| | 1.89-2.00 (m, 2H), 1.69-1.88 (m, 2H) 1.42-1.54 (m, 1H). | | |
| Target 8 | ET56634-26-P1A (400 MHz, CDCl₃) | 16.14 | 10.0% |
| | δ 6.89-6.98 (m, 3H), 6.83-6.88 (m, 2H), 6.24 (q, J = 2.8 Hz, 1H), | | |
| | 5.27 (s, 1H), 3.90 (d, J = 7.2 Hz, 6H), 3.24 (brs, 1H), 2.80 (brs, | | |
| | 1H), 2.40 (s, 3H), 2.24-2.35 (m, 2H), 2.06-2.24 (m, 2H), 1.65- | | |
| | 1.98 (m, 4H), 1.37-1.51 (m, 1H). | | |
| Target 9 | ET56634-22-P1A (400 MHz, CDCl₃) | 24.04 | 20.3% |
| | δ 6.82-6.92 (m, 3H), 5.74-5.82 (m, 2H), 5.41-5.45 (m, 1H), 3.90 | | |
| | (s, 3H), 3.87 (s, 3H), 3.36-3.40 (m, 1H), 2.69 (s, 1H), 2.56-2.63 | | |
| | (m, 5H), 2.44-2.46 (m, 4H), 2.13-2.20 (m, 2H), 1.65-1.71 (m, | | |
| | 1H). | | |
| Target 10 | ET56634-23-P1A (400 MHz, CDCl₃) | 23.88 | 20.3% |
| | δ 6.82-6.93 (m, 3H), 5.78 (s, 2H), 5.42-5.46 (m, 1H), 3.90 (s, | | |
| | 3H), 3.88 (s, 3H), 3.32-3.36 (m, 1H), 2.38-2.54 (m, 10H), 2.06- | | |
| | 2.17 (m, 2H), 1.95-1.99 (m, 2H), 1.59-1.62 (m, 1H). | | |
| Target 11 | ET56634-35-P1A1 (400 MHz, MeOD) | 21.24 | 19.6% |
| | δ 6.90-7.13 (m, 3H), 5.99-6.14 (m, 2H), 5.69 (t, J = 7.8 Hz, 1H), | | |
| | 3.91 (s, 2H), 3.79-3.83 (m, 8H), 3.45-3.56 (m, 1H), 3.05 (s, 3H), | | |
| | 2.80-2.92 (m, 1H), 2.55-2.65 (m, 1H), 2.26-2.35 (m, 1H), 1.92- | | |
| | 2.05 (m, 1H). | | |
| Target 12 | ET56634-36-P1B (400 MHz, CDCl₃) | 24.41 | 16.9% |
| | δ 6.91-7.11 (m, 3H), 5.98-6.18 (m, 2H), 5.57-5.73 (m, 1H), 4.17 | | |
| | (q, J = 7.2 Hz, 1H), 3.78-3.87 (m, 8H), 3.46-3.55 (m, 1H), 3.06 | | |
| | (s, 3 H), 2.82-2.89 (m, 1 H), 2.57-2.67 (m, 1H), 2.26-2.37 (m, | | |
| | 1H), 1.91-2.02 (m, 1H), 1.54 (d, J = 7.6 Hz, 3H). | | |
| Target 13 | ET56634-30-P1A (400 MHz, CDCl₃) | 18.86 | 23.8% |
| | δ 6.87-6.96 (m, 2H), 6.83 (d, J = 8.4 Hz, 1H), 5.72-5.81 (m, 2H), | | |
| | 5.37-5.46 (m, 1H), 3.89 (d, J = 10.8 Hz, 6H), 3.27 (s, 1H), 2.26- | | |
| | 2.49 (m, 8H), 2.01-2.24 (m, 2H), 1.48-1.53 (m, 1H), 1.16 (t, J = | | |
| | 7.6 Hz, 3H). | | |
| Target 14 | ET56634-3-P1A (400 MHz, CDCl₃) | 21.60 | 34.7% |
| | δ 6.88-6.95 (m, 2H), 6.81-6.86 (m, 1H), 5.72-5.83 (m, 2H), 5.41 | | |
| | (dd, J = 10.4, 5.6 Hz, 1H), 3.89 (d, J = 10.4 Hz, 6H), 3.27 (t, J = | | |
| | 8.0 Hz, 1H), 2.52-2.59 (m, 1H), 2.30-2.47 (m, 6H), 2.02-2.22 (m, | | |
| | 2H), 1.50 (t, J = 12.0 Hz, 2H), 1.18 (d, J = 6.4 Hz, 6H), 0.80-0.92 | | |
| | (m, 1H). | | |
| Target 15 | ET56634-4-P1A (400 MHz, CDCl₃) | 21.76 | 28.2% |
| | δ 8.05-8.10 (m, 2H), 7.53-7.60 (m, 1H), 7.42-7.49 (m, 2H), 6.91- | | |
| | 6.98 (m, 2H), 6.85 (d, J = 8.0 Hz, 1H), 5.82-5.95 (m, 2H), 5.65- | | |
| | 5.69 (m, 1H), 3.90 (d, J = 12.4 Hz, 6H), 3.24-3.34 (m, 1H), 2.27- | | |
| | 2.52 (m, 7H), 2.05-2.19 (m, 1H), 1.67 (t, J = 7.6 Hz, 1H), 0.80- | | |
| | 0.89 (m, 1H). | | |
| Target 16 | ET56634-29-P1A (400 MHz, CDCl₃) | 21.35 | 21.3% |
| | δ 6.90-6.98 (m, 1H), 6.85 (d, J = 8.0 Hz, 1H), 6.25-6.30 (m, 1H), | | |
| | 5.79-5.92 (m, 1H), 5.59-5.63 (m, 1H), 3.90 (d, J = 12.0 Hz, 2H), | | |
| | 3.29 (t, J = 8.4 Hz, 1H), 2.32-2.50 (m, 2H), 2.23-2.30 (m, 1H), | | |
| | 2.05-2.17 (m, 1H), 1.55-1.71 (m, 1H). | | |

### Conclusion

All sixteen target compounds were prepared and tested in good quantities and yields using the synthetic methods described above.

### Example 5: In Vitro Pharmacology Inhibition Assays

SERT assay (Compounds of Formula I):

Test compounds (conc range), reference compound or vehicle control were incubated for 180 min at room temperature with CHO cells stably transfected with the human serotonin transporter (5×10³ cells/well) with 0.15 μM [3H] serotonin in the presence or absence of the test or reference compound in buffer containing (in mM); Tris/HCl (pH 7.4) (5), HEPES/Tris (7.5), NaCl (120), KCl (5.4), CaCl₂ (1.2), MgSO₄ (1.2), Glucose (5) and ascorbic acid (1). Following incubation, [³H] serotonin uptake was quantified by a standard scintillation counting method.

SERT assay (Compounds of Formula II):

SERT assays were performed using a standard transporter uptake assay kit, (Molecular devices, (R8174)). In short, test compounds (10 nM-30 μM), reference compound or vehicle control were added to cells stably expressing hSERT (20,000/well) in assay buffer containing HBSS (20 mM) and BSA (0.1% (w/v)) for 30 mins at 30° C. Assay plates were incubated in the presence of dye for a further 60 mins at 37° C. and intracellular fluorescence intensity was determined.

### PDE4-B1 & PDE4-D2 Assays (Compounds of Formula I):

Test compounds (conc range), reference compound or vehicle control were added to assay buffer containing (in mM); Tris/HCl, pH 7.4 (40), MgCl₂ (8) and EGTA/NaOH (1.7), containing 450 nM cAMP (450 nM) and 0.25 μCi (PDE-4B1) or 0.0125 μCi (PDE-4D2) [³H] cAMP. 20 mins after addition of human recombinant PDE-4B1 (1.2 IEU) or PDE-4D2 (1.5 IEU), SPA beads were added and incubated at 22° C. for a further 30 mins. [³H]5′AMP was quantified by a standard scintillation counting method.

PDE-41A assay (Compounds of Formula II):

PDE-41A assays were performed using the AMP-Glo™ Assay Kit, (Promega-V5012) human recombinant PDE-4B1A (Sigma, SRP0262) according to recommended protocols. In short, test compounds (10 nM-30 μM), reference compound or vehicle control were added to PDE-41A (0.015 nM) for 20 mins at 23° C. after which, separated by 60 mins, cAMP (1.2 nM), and cAMP detection solution were added to the assay and luminescence (AMP generation) was determined.

### Statistical Analysis:

Data were expressed as the mean of two technical replicates for each data point, and percent inhibition calculated relative to maximum inhibition observed in the presence of the reference comparator Ro 20-1724 for PDE-4 assays, and imipramine for serotonin assays. Where at least three concentrations were examined, IC₅₀ values were derived from non-linear regression, 4-parameter logistic fit of log 10 concentration effect curves for each compound using GraphPad Prism software and are summarized in the tables below. Where 50% inhibition was not achieved, the IC₅₀ value was set at greater than the highest concentration examined.

### Results

Table 5 below details the compounds that were tested at more than three concentrations and as such detail the I050 values that were derived.

TABLE 5
Potency determinations for compounds
of Formula (I) at SERT and PDE-4

| Compound ID no from | IC₅₀ (μM) | | |
|---|---|---|---|
| Table 1 | SERT | PDE-4B1 | PDE-4D2 |
| 1 | 0.1 | 1 | 0.1 |
| 2 | 0.03 | 0.9 | 0.2 |
| 3 | 0.005 | 5.2 | 1.3 |
| 4 | <0.1 | 7.1 | 11.3 |
| 6 | 0.002 | 0.7 | 0.2 |
| 7 | 0.02 | 1.3 | 0.5 |
| 8 | 0.4 | 4.5 | 3.3 |
| 9 | <0.1 | 13.9 | 1.2 |
| 10 | 0.3 | >30 | >30 |
| 11 | 0.8 | 0.6 | 0.1 |
| 12 | >10 | 2.4 | 4.3 |
| 13 | 0.02 | 1 | 0.5 |
| 15 | <3 | <3 | <3 |
| 21 | 0.03 | 1.7 | 1 |
| 24 | 0.02 | 2.3 | 1.4 |
| 25 | 0.3 | >30 | >30 |
| 27 | 0.4 | 1.2 | 0.6 |
| 31 | <0.1 | 15.2 | 8.12 |
| 33 | 2.1 | >30 | >30 |
| 34 | 0.9 | >30 | >30 |
| 35 | 0.2 | >30 | >30 |
| 36 | 0.4 | >30 | >30 |
| 37 | 0.8 | >30 | >30 |
| 38 | 0.4 | >30 | >30 |
| 39 | 4.5 | >30 | >30 |
| 40 | >3 | >3 | >3 |
| 41 | <3 | >3 | >3 |
| 42 | >3 | >3 | <3 |
| 43 | >3 | >3 | >3 |
| 44 | >3 | <3 | <3 |
| 45 | <3 | >3 | <3 |

IC50—inhibitory concentration at 50% level. Represents that determined by 4-parameter logistical fit, two technical replicates per concentration examined at between 10 nM and 10 μM. NH ranges from 0.7-1.1. No constraints imposed on logistical fit.
SERT—serotonin transporter; PDE-4—Phosphodiesterase-4.

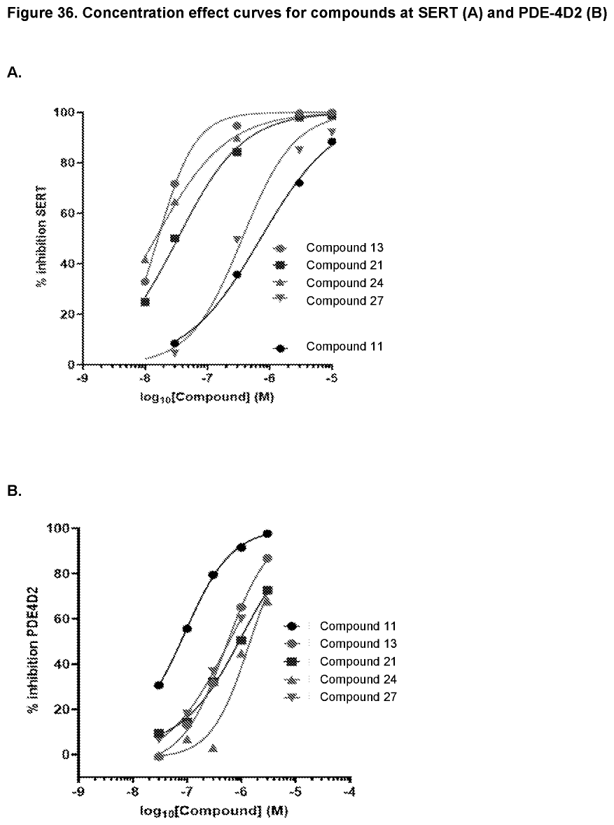

FIG. 36 details the concentration effect curves for compound numbers 11, 13, 21, 24 and 27 at SERT (A) and PDE-41D2 (B).

Some of the compounds of Formula (1) were only tested at a single concentration and as such the data for these compounds is represented below in Table 6.

TABLE 6
Percentage inhibition for compounds
of Formula (I) at SERT and PDE-4

| | % SERT | % PDE-4B1 | % PDE-4D2 |
|---|---|---|---|
| Compound ID no from | inhibition | inhibition | inhibition |
| Table 1 | (10 μM) | (30 μM) | (30 μM) |
| 5 | 98 | 88 | 94 |
| 13 | 100 | 96 | 98 |
| 14 | 59 | 94 | 97 |
| 16 | 98 | −1 | 3 |
| 17 | 99 | 61 | 74 |
| 18 | 86 | −4 | −3 |
| 19 | 99 | 1 | 3 |
| 20 | 100 | 83 | 92 |
| 21 | 91 | 95 | 95 |
| 22 | 91 | 38 | 37 |
| 23 | 77 | 88 | 93 |
| 24 | 91 | 89 | 95 |
| 26 | 81 | 26 | 20 |
| 27 | 96 | 96 | 99 |
| 28 | 98 | 53 | 55 |
| 29 | 99 | 40 | 32 |
| 30 | 95 | 24 | 31 |

Table 7 details the compounds of Formula (II) which were tested at two different concentrations in the SERT and PDE-4A1A inhibition assays. Table 7. Percentage inhibition for compounds of Formula (II) at SERT and PDE-4

TABLE 7
Percentage inhibition for compounds
of Formula (II) at SERT and PDE-4

| Compound ID | % SERT | % SERT | % PDE-4A1A | % PDE-4A1A |
|---|---|---|---|---|
| no from | inhibition | inhibition | inhibition | inhibition |
| Table 1 | (1 μM) | (3 μM) | (30 μM) | (100 μM) |
| 46 | 81 | 94 | 53 | 79 |
| 47 | 90 | 94 | 40 | 88 |
| 48 | 93 | 101 | −8 | 48 |
| 49 | 95 | 97 | 87 | 86 |
| 50 | 98 | 98 | 92 | 101 |
| 51 | 99 | 98 | 95 | 99 |
| 52 | 91 | 91 | 97 | 99 |
| 53 | 99 | 98 | 94 | 97 |
| 54 | 78 | 98 | 98 | 101 |
| 55 | 67 | 97 | 100 | 95 |
| 56 | 86 | 99 | 74 | 102 |
| 57 | 86 | 97 | 101 | 102 |
| 58 | 98 | 98 | 103 | 100 |
| 59 | 99 | 97 | 100 | 100 |
| 60 | 92 | 95 | 98 | 100 |
| 61 | 101 | 106 | 94 | 90 |

### Conclusion

The in vitro data detailed in Example 1 demonstrates the ability of the compounds of the invention to inhibit both the serotonin transporter (SERT) and phosphodiesterase-4 (PDE-4).

Inhibition of SERT is demonstrated by nearly all antidepressants to varying degrees. Inhibiting SERT increases the amount serotonin available in the synapse, which in turn leads to downstream cellular and molecular adaptations that are thought to mediate the efficacy of medications which are used as antidepressants.

Phosphodiesterase-4 (PDE-4) is mainly present in immune cells, epithelial cells, and brain cells. It manifests as an intracellular non-receptor enzyme that modulates inflammation and epithelial integrity. A compound's ability to inhibit PDE-4 is suggestive that they may serve as a promising therapeutic target for the treatment of diverse pulmonary, dermatological, and severe neurological diseases.

As such the compounds of Formula (I) and Formula (II) may prove to be useful in the treatment of diseases and conditions which benefit from inhibition of SERT and/or PDE-4. Such diseases include the prevention or treatment of depression, anxiety, inflammation, and/or autoimmunity.

### Example 6: Pharmacokinetic Study in Mice

### Methods:

Groups of 18 male C57Bl/6J mice (weighing between 22.7 and 27.3 g) received a single administration of test compound (30 mg/kg; i.p.) in methylcellulose (0.5% w/v) at a nominal concentration of 3.0 ng/ml.

Three mice from each dose group were subject to cardiac puncture under general anesthesia at 0.25, 0.50, 1.00, 1.50, 2.00 and 4.00 hours post-dose and plasma samples generated for application of standard LC-MS/MS bioanalytical methods.

Quantitation of compound concentration was derived from reference calibration data. Pharmacokinetic data were derived from serial plasma concentrations.

### Results:

Table 8 details the PK parameters measures using six different test compounds of the invention.

TABLE 8
Summary of PK parameters

| Compound ID | Cₘₐₓ | Tₘₐₓ | AUC0-inf | t_{1/2} |
|---|---|---|---|---|
| no from Table 1 | (ng/mL) | (hr) | (ng · hr/mL) | (hr) |
| 6 | 2606 | 0.25 | 1956 | 0.56 |
| 11 | 796 | 0.25 | 1041 | 0.94 |
| 13 | 2333 | 0.25 | 2100 | 0.67 |
| 21 | 2426 | 0.25 | 1834 | 0.57 |
| 24 | 492 | 0.25 | 825 | 1.28 |
| 27 | 831 | 0.25 | 1398 | 0.97 |

### Conclusion

All six compounds tested demonstrated a short Tₘₐₓ at only 15 minutes and relatively short half-lives ranging from half an hour to 1.3 hours. These effects are indicative of medications that will take effect quickly and be cleared from the body in a short space of time also.

Different Cₘₐₓ and AUCs were found for the various different compounds. These ranged from around 500 ng/ml to over 2500 ng/ml for Cₘₐₓ and 825 ng/hr/ml to 2100 ng/hr/ml for AUC. The finding of such a variation in pharmacokinetic parameters for the different compounds was surprising given that all of the compounds are derived from a mesembrine alkaloid.

This demonstrates that alteration of different parts of the scaffold can produce an alteration in the characteristics of the compound.

### Example 7: Hepatocyte Stability Study

### Methods:

Two different compounds, namely mesembrine and deuterated mesembrine, as identified as compound 5 from Table 1, were tested in the hepatocyte stability study. Compound 5 is deuterated mesembrine and as such it was important to determine whether deuteration had an effect of the metabolism of the compound.

The compounds were added at a concentration of 1 μM to human hepatocytes (0.5×10⁶ cells/ml in Williams E media supplemented with 2 mM L-glutamine and 25 mM HEPES) and incubated for 0, 5, 10, 20, 40 and 60 min at 37° C. Five replicates were performed for each compound.

Lysates were obtained by centrifugation at 3,000 rpm for 30 min at 4° C. and analysed according to generic LC/MS/MS methods to calculate intrinsic clearance (CLᵢₙₜ), standard error (SE CLᵢₙₜ), and t_{1/2}.

### Results:

Table 9 below details the results from the two compounds in the hepatocyte stability study.

TABLE 9
Hepatocyte stability study

| Compound ID no | Qualified Clᵢₙₜ | Qualified | |
|---|---|---|---|
| from Table 1 | (μl/min/10⁶ cells) | SE Clᵢₙₜ | Qualified t_{1/2} |
| Mesembrine | 16.3 | 0.7 | 84.8 |
| 5 | 21.1 | 2.6 | 65.8 |

### Conclusion

The deuterated compound 5 was found to have a shorter half-life than the non-deuterated compound mesembrine. This suggests that deuteration of mesembrine increases the rate of clearance from human hepatocytes.

Such a finding is surprising given that drugs are commonly deuterated in the pharmaceutical industry to increase the half-life of the medication.

### Example 8: Open Field Test

The open field test is routinely applied in order to assess a compounds ability to affect anxiety, and as such as positive result may be indicative of a compounds anxiolytic activity (Kraeuter et al., 2019).

### Methods:

Male C57Bl/6J mice (3-4 months), were assigned to one of three groups receiving either test compound (0.3, 3.0 mg/kg) or vehicle (0.5% (w/v) methyl cellulose) via the i.p. route, 60 minutes before experimental examination.

The test compounds used were compound identification numbers 6, 13 and 21 from Table 1.

The duration of time the mouse spent in central, periphery and corners were quantified, with duration in the central area depicted as the primary outcome measure.

Data were tested for normality and outliers were removed. For analysis of parametric data one way ANOVA analysis followed by Dunnett's post hoc comparisons was used, whereas for the analysis of non-parametric data Kruskal-Wallis test with Dunn's correction was used to determine statistical difference between the effect of the compound on the primary endpoint when compared to the vehicle control group.

### Results:

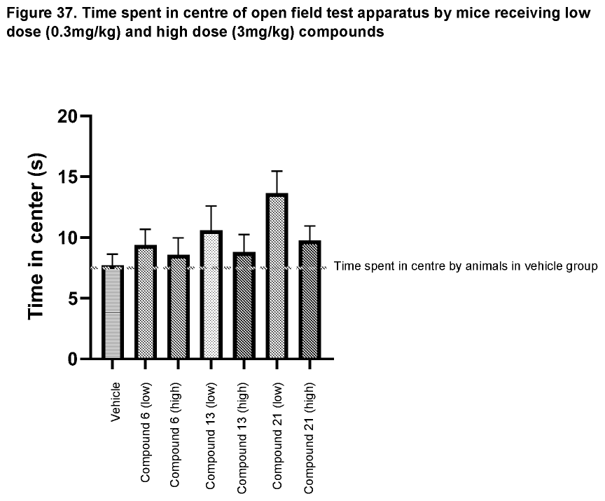

Table 10 and FIG. 37 detail the time spent by the mice in each group in the centre of the apparatus during the study.

TABLE 10
Time spent in centre during open field test

| Compound ID no | Time (s) | Time (s) |
|---|---|---|
| from Table 1 | Low dose (0.3 mg/kg) | High dose (3 mg/kg) |
| Vehicle | 7.7 | 7.7 |
| 6 | 9.4 | 8.6 |
| 13 | 10.6 | 8.8 |
| 21 | 13.7 | 9.8 |

As can be seen all three compounds at both the low and high dose increased the time the mouse spent in the centre of the open field apparatus compared to the mice which were administered vehicle.

Statistical analysis showed that compound 21 administered at the low dose of 0.3 mg/kg produced a statistically significant increase in the time spent in the centre compared to vehicle.

### Conclusion

The numerical increase in time spent in exploratory behaviour, in the centre of the open field, are indicative of the ability of the compounds tested to produce an anxiolytic effect compared to vehicle.

Such a finding is suggestive that the compounds of the invention may be useful in the prevention or treatment of conditions or diseases such as depression or anxiety.

### Example 9: Forced Swim Test

The forced swim test is a standard model for examination of potential antidepressant-like activity and represents futility, despair, and motivation behavioural domains.

### Methods:

Male C57Bl/6J mice (3-4 months), were assigned to one of three groups receiving either test compound (0.3, 3.0 mg/kg) or vehicle (0.5% (w/v) methyl cellulose) via the i.p. route, 60 minutes before experimental examination.

The test compounds used were compound identification numbers 6, 13 and 21 from Table 1.

The duration of time the mouse spent immobile during the forced swim test was measured.

Data were tested for normality and outliers were removed. For analysis of parametric data one way ANOVA analysis followed by Dunnett's post hoc comparisons was used, whereas for the analysis of non-parametric data Kruskal-Wallis test with Dunn's correction was used to determine statistical difference between the effect of the compound on the primary endpoint when compared to the vehicle control group.

### Results:

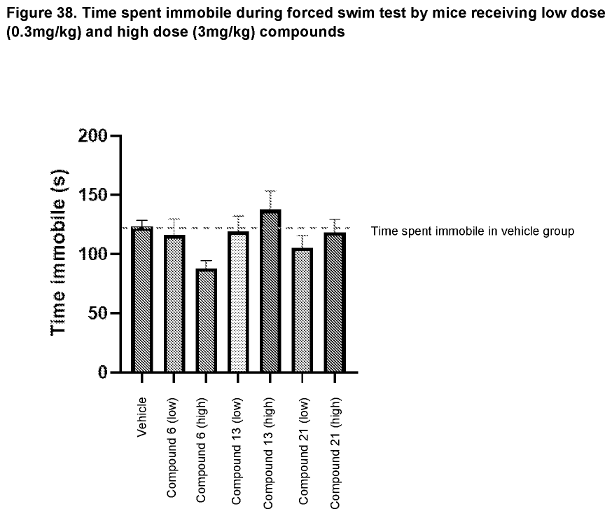

Table 11 and FIG. 38 detail the time spent immobile by the mice in each group during the study.

TABLE 11
Time spent immobile during forced swim test

| Compound ID no | Time (s) | Time (s) |
|---|---|---|
| from Table 1 | Low dose (0.3 mg/kg) | High dose (3 mg/kg) |
| Vehicle | 123 | 123 |
| 6 | 116 | 88 |
| 13 | 119 | 138 |
| 21 | 105 | 118 |

As can be seen all three compounds at both the low and high dose, except the high dose compound 13, decreased the time the mouse spent immobile in comparison to the mice which were administered vehicle.

### Conclusion

The numerical decrease in time spent immobile, are indicative of the ability of the compounds tested to produce an anti-depressant effect compared to vehicle.

Such a finding is suggestive that the compounds of the invention may be useful in the prevention or treatment of conditions or diseases such as depression or anxiety.

### Example 10: Modulation of LPS-Induced Cytokine Release from Human Peripheral Blood Mononuclear Cells (PBMCs)

Human peripheral blood mononuclear cells (PBMCs) are immune cells with a single, round nucleus that originate in bone marrow and are secreted into the peripheral circulation. These cells are critical components of the immune system and are involved in both humoral and cell-mediated immunity.

The ability of a compound to modulate the release of cytokines can be used to determine the proficiency of the compound to prevent or treat diseases or conditions associated with inflammation or auto-immunity.

### Methods:

Venous blood from healthy human donors was collected in K2 EDTA vacuum tubes, mixed (1:1) with sterile PBS and PBMCs were isolated using SepMate tubes by centrifugation.

PBMCs were seeded in 24 well plates at a density of 0.5×10⁶ cells/well in serum free RPMI media.

PBMCs were exposed to each of four test compounds, compounds 1, 2, 11 and 27 as identified in Table 1 at three different concentrations (0.1, 1.0, or 10 mg/kg) for 45 mins, before treatment with LPS for 24 hrs.

Supernatants were obtained by centrifugation and subject to cytokine quantification, using the MESO QuickPlex SQ 120 where a panel of cytokines (TNF-α, IL-1β, and IL-10) were analysed using a Human V-Plex Proinflammatory panel multiplex assay.

Cytokine concentrations were expressed as the mean of a single data point from each of three donors and percent LPS response relative to vehicle as well as percent inhibition calculated relative to maximal response to LPS.

Ratios of IL-10 to TNF-α of the percent LPS response normalized to the vehicle LPS group were additionally calculated.

Data were tested for normality. For analysis of parametric data, one way ANOVA analysis followed by Dunnett's post hoc comparisons was used, whereas for the analysis of non-parametric data Kruskal-Wallis test with Dunn's correction was used to determine statistical difference between the effect of compound on the endpoints when compared to the vehicle LPS group. IC₅₀ values were derived from non-linear regression, 4-parameter logistic fit of log 10 concentration effect curves for each compound using GraphPad Prism software Results:

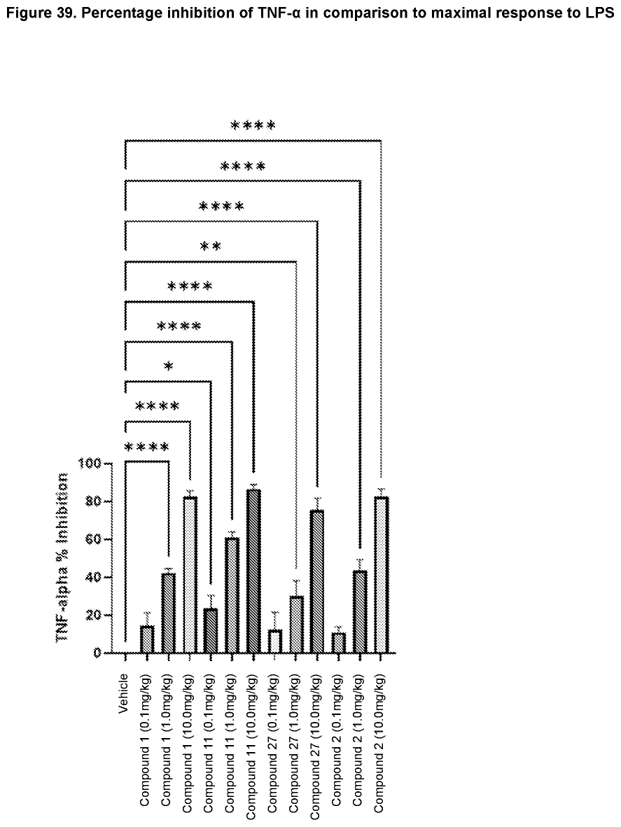

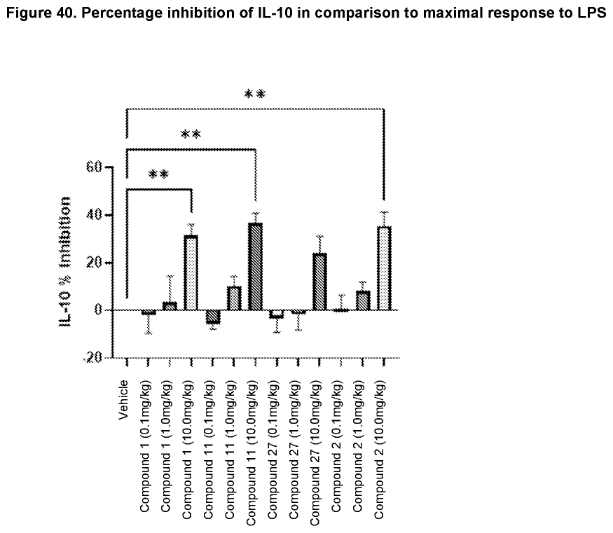

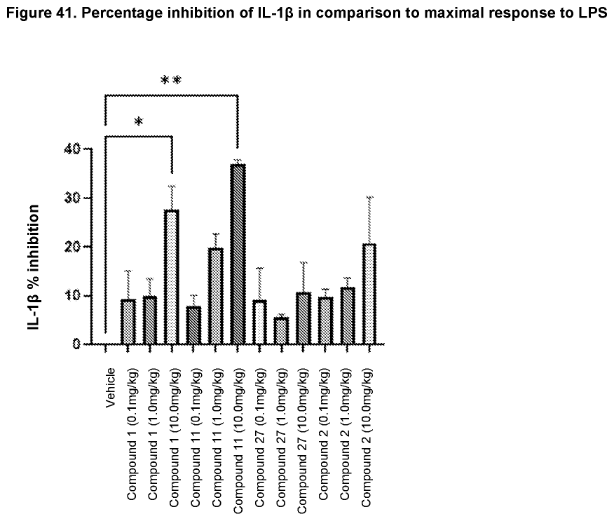

FIGS. 39, 40 and 41 detail the percentage inhibition of the various cytokines, TNF-α, IL-1β, and IL-10, relative to the maximal response to LPS.

As can be seen compounds 1, 2 and 27 at the medium (1.0 μM) and high (10.0 μM) concentration inhibited TNF-α release at a statistically significant level compared to vehicle.

In addition, compound 11 at all concentrations was able to inhibit TNF-α release at a statistically significant level compared to vehicle.

Inhibition of IL-10 was statistically significantly inhibited by the high concentration (10.0 μM) compounds 1, 2 and 11.

Inhibition of IL-β was statistically significantly inhibited by the high concentration (10.0 μM) compounds 1 and 11.

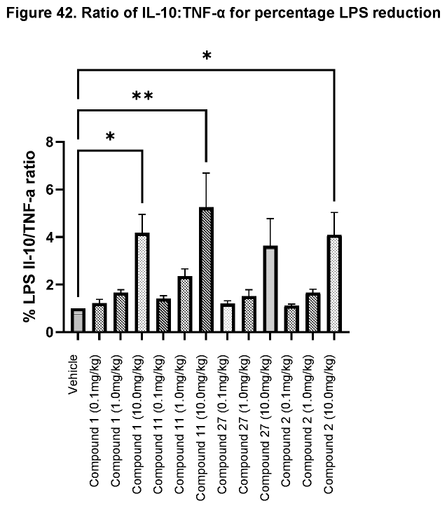

FIG. 42 and Table 12 provide details of the ratio of IL-10:TNF-α. Compounds 1, 2 and 11 at the highest concentration tested (10 μM) produced a statistically significant increase in the ratio of IL-10:TNF-α.

TABLE 12
Ratio of IL-10:TNF-α

| | | Com- | Com- | Com- | Com- |
|---|---|---|---|---|---|
| Concentration | | pound | pound | pound | pound |
| (μM) | Vehicle | 1 | 11 | 27 | 2 |
| 0.1 | 1 | 1.2 | 1.4 | 1.2 | 1.1 |
| 1 | 1 | 1.7 | 2.4 | 1.5 | 1.7 |
| 10 | 1 | 4.2 | 5.3 | 3.6 | 4.1 |

### Conclusion

The ability of the test compounds to inhibit the release of the inflammatory cytokines is suggestive of their potential for use in the prevention or treatment of diseases or conditions associated with inflammation and/or autoimmunity.

Furthermore, the ability of the compounds to increase the ratio of IL-10:TNF-α is of significance as it is demonstrative of the compounds potential for use in the treatment of a range of inflammatory diseases.

For example, in cardiac failure the change in the ratio of IL-10 to TNF-α is more predictive than changes in TNF-α alone. The ability of the compounds of the invention to increase the ratio could enable their utility in the treatment of a variety of diseases and conditions.

### Embodiments

1. A compound of Formula (I) or a salt thereof,

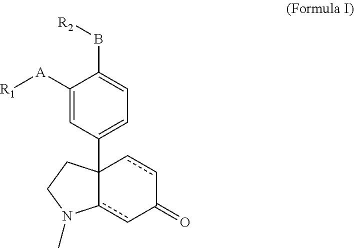

Wherein:
[symbol] is a single or double bond, provided that there are no more than one double bond;
A and B are independently O or S;
R1 and R2 are independently selected from the group consisting of:
C1-C6 alkyl, C2-C6 alkenyl, C2-C6 alkynyl, 3- to 8-membered carbocycle, 3- to 8-membered heterocycle, hydrogen, deuterium and halogen, and wherein A and B are O, R¹ and R² are not mutually a C1 alkyl or not mutually a hydrogen.

2. The compound of embodiment 1, wherein A is O or S.

3. The compound of embodiment 1 or 2, wherein B is O.

4. The compound of any one of embodiments 1-3, wherein R1 and R2 are independently selected from the group consisting of: C1-C6 alkyl optionally substituted with one or more deuterium, —C1-C6alkylene-3-to 8-membered carbocycle, C1-C6 haloalkyl, C2-C6 alkenyl, C2-C6 alkynyl, 3- to 8-membered carbocycle, 3- to 8-membered heterocycle, hydrogen, deuterium, and halogen.

5. The compound of any one of embodiments 1-4, wherein R1 is C1-6alkylene-C3-8cycloalkyl, C1-C6 alkyl optionally substituted with one or more deuterium, C1-C6haloalkyl, hydrogen, 3- to 8-membered cycloalkyl, C1-6alkylene-phenyl, C2-C6 alkenyl, or C2-C6 alkynyl.

6. The compound of embodiment 5, wherein R1 is —CH₂-cyclopropyl, —CH₂CH₂CH₂CH₃, —CH₂CH₃, —CH₂CF₃, —CD₃, —CH₃, —CH₂CHF₂, hydrogen, cyclopentyl, —CH₂-phenyl, —CH₂—CH═CH₂, cyclohexyl, or —CH₂—C≡CH.

7. The compound of any one of embodiments 1-6, wherein R2 is C1-C6 alkyl or C1-6alkylene-C3-8cycloalkyl.

8. The compound of embodiment 7, wherein R2 is —CH₃, —CH₂CH₃, —CH(CH₃)₂, or —CH₂— cyclopropyl.

9. The compound of any one of embodiments 1-8, wherein one or more [symbol] is a single bond.

10. The compound of any one of embodiments 1-8, wherein one [symbol] is a double bond.

11. A compound of Formula (II), or a salt thereof,

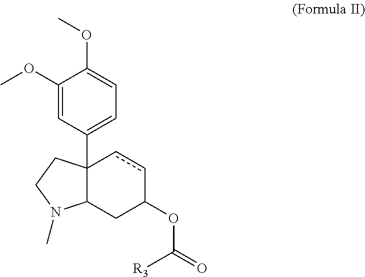

Wherein:
[symbol] is a single or double bond;
R3 is selected from the group consisting of:
C1-C6 straight-chain or branched-chain alkyl, C1-C6 alkyl carboxylic acid, C1-C6 straight chain or branched chain amide, benzyl, phenyl, or pyrrole.

12. The compound of embodiment 11, wherein [symbol] is a single bond.

13. The compound of embodiment 11, wherein [symbol] is a double bond.

14. The compound of any one of embodiments 11-13, wherein R3 is C2-C6 alkyl-carboxylic acid, pyrrole, or C1-C6 straight-chain or branched-chain alkyl optionally substituted with an amino.

15. The compound of embodiment 14, wherein R3 is —CH₂CH₂—CO₂H, —CH₂CH₂CH₂—CO₂H, —CH₂NH₂, —CH(CH₃)NH₂, —CH₂CH₃, —CH(CH₃)₂, or pyrrole.

16. A compound of Formula (I) or Formula (II) as defined by any one of the compounds numbered 1 to 61 in Table 1.

17. A pharmaceutical composition comprising a compound of Formula (I) or Formula (II), or a salt thereof, together with one or more ingredients selected from carriers, diluents, excipients, adjuvants, fillers, buffers, binders, disintegrants, preservatives, antioxidants, lubricants, stabilisers, solubilisers, surfactants (e.g., wetting agents), masking agents, colouring agents, flavouring agents, and sweetening agents.

18. A compound of Formula (I) or Formula (II), or the pharmaceutical composition comprising the compound of Formula I or Formula II, for use as a medicament.

19. A compound of Formula (I) or Formula (II), or the pharmaceutical composition comprising the compound of Formula I or Formula II, for use in the prevention or treatment of disease or condition associated with depression, anxiety, inflammation, and/or autoimmunity.

20. A compound of Formula (I) or Formula (II), or the pharmaceutical composition comprising the compound of Formula I or Formula II, for use according to embodiment 18, wherein the prevention or treatment is provided for the group consisting of: addiction, alcoholism, Alzheimer's disease, anxiety, attention deficit disorder (ADHD), binge eating, cluster headaches, complicated grief disorder, depression and anxiety associated with terminal illness, depressive and anxiety disorders, erectile dysfunction, hypersomnia, improving mood in healthy human subjects; insomnia, irritable bowel syndrome, major depression, mania, mental disorders, migraine headaches, pain, panic disorders, Parkinson's disease, post-traumatic mania, post-traumatic stress disorder (PTSD), premature ejaculation, prolonged grief disorder, psychosis, terminal illness, Tourette's syndrome, treatment resistant anxiety, and treatment resistant depression.

21. A compound of Formula (I) or Formula (II), or the pharmaceutical composition comprising the compound of Formula I or Formula II, for use according to embodiment 18, wherein the prevention or treatment is provided for the group consisting of: acne vulgaris; acute inflammation; Addison's disease; allergic reactions; allergies; Alzheimer's disease; ankylosing spondylitis; aplastic anemia; asthma; atherosclerosis; autoimmune vasculitis; cancer; celiac disease; chronic inflammatory demyelinating polyneuropathy (CIDP); chronic obstructive pulmonary disease (COPD); colitis; diverticulitis; endometriosis; familial Mediterranean fever; fatty liver disease; glomerulonephritis; Grave's disease; Guillain-Barre syndrome; Hashimoto's thyroiditis; headaches, including chronic headaches and migraine; hemolytic anemia; hidradenitis suppurativa; HIV and AIDS; hypersensitivity reactions; immune-mediated inflammatory disease (IMID); inflammatory bowel disease such as Crohn's disease and ulcerative colitis; inflammatory myopathies; interstitial cystitis; leukocyte defects; lichen planus; mast cell activation syndrome; mastocytosis; mental health conditions where inflammation and/or autoimmunity is a co-morbid or causative factor, including; depression, schizophrenia, and anxiety; multiple sclerosis; myasthenia gravis; obesity; otitis; pain, including acute and chronic pain; Parkinson's disease; pelvic Inflammatory disorder; peripheral ulcerative keratitis; pernicious anemia; pharmacological inflammatory response; pneumonia; prostatitis; psoriasis; psoriatic arthritis; reperfusion injury; rheumatic fever; rheumatoid arthritis; rhinitis; sarcoidosis; scleroderma; Sjogren's syndrome; systemic lupus erythematosus (SLE); transplant rejection syndrome; type I diabetes; type II diabetes; vasculitis; and vitiligo.

22. A method of treatment comprising administering to a subject in need thereof a therapeutically effective amount of a compound of Formula (I) or Formula (II) or salt thereof.

23. A method of synthesizing a compound of Formula (I) or Formula (II).

24. An intermediate formed in the method of synthesis of the compound of Formula (I) or Formula (II).

25. An intermediate formed in the method of synthesis of the compound of Formula (I) or Formula (II) as claimed in embodiment 23, wherein the intermediate is a bromine intermediate, as defined by compounds 8 or 9 in Table 1.

1. A compound of Formula (I) or a salt thereof, 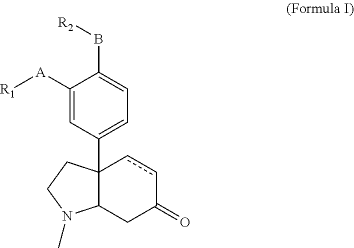
Wherein: [symbol]
is a single or double bond;
A and B are independently O or S;
R1 and R2 are independently selected from the group consisting of:
C1-C6 alkyl, C2-C6 alkenyl, C2-C6 alkynyl, 3- to 8-membered carbocycle, 3- to 8-membered heterocycle, hydrogen, deuterium or halogen, and
wherein A and B are O, R¹ and R² are not mutually a C1 alkyl or not mutually a hydrogen.

2. A compound of Formula (II), or a salt thereof, 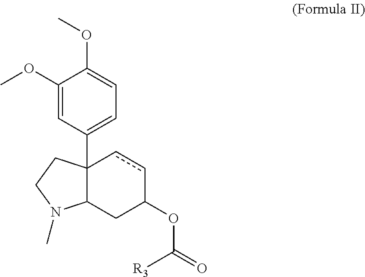
Wherein: [symbol]
is a single or double bond;
R3 is selected from the group consisting of:
C1-C6 straight-chain or branched-chain alkyl, C1-C6 alkyl carboxylic acid, C1-C6 straight chain or branched chain amide, benzyl or pyrrole.

3. A compound of Formula (I) or Formula (II) as defined by any one of the compounds numbered 1 to 61 in Table 1.

4. A pharmaceutical composition comprising a compound of Formula (I) or Formula (II), or a salt thereof, together with one or more ingredients selected from carriers, diluents, excipients, adjuvants, fillers, buffers, binders, disintegrants, preservatives, antioxidants, lubricants, stabilisers, solubilisers, surfactants (e.g., wetting agents), masking agents, colouring agents, flavouring agents, and sweetening agents.

5. A compound of Formula (I) or Formula (II), or the pharmaceutical composition comprising the compound of Formula I or Formula II, for use as a medicament.

6. A compound of Formula (I) or Formula (II), or the pharmaceutical composition comprising the compound of Formula I or Formula II, for use in the prevention or treatment of disease or condition associated with depression, anxiety, inflammation, and/or autoimmunity.

7. A compound of Formula (I) or Formula (II), or the pharmaceutical composition comprising the compound of Formula I or Formula II, for use according to claim 5, wherein the prevention or treatment is provided for the group consisting of: addiction, alcoholism, Alzheimer's disease, anxiety, attention deficit disorder (ADHD), binge eating, cluster headaches, complicated grief disorder, depression and anxiety associated with terminal illness, depressive and anxiety disorders, erectile dysfunction, hypersomnia, improving mood in healthy human subjects; insomnia, irritable bowel syndrome, major depression, mania, mental disorders, migraine headaches, pain, panic disorders, Parkinson's disease, post-traumatic mania, post-traumatic stress disorder (PTSD), premature ejaculation, prolonged grief disorder, psychosis, terminal illness, Tourette's syndrome, treatment resistant anxiety, and treatment resistant depression.

8. A compound of Formula (I) or Formula (II), or the pharmaceutical composition comprising the compound of Formula I or Formula II, for use according to claim 5, wherein the prevention or treatment is provided for the group consisting of: acne vulgaris; acute inflammation; Addison's disease; allergic reactions; allergies; Alzheimer's disease; ankylosing spondylitis; aplastic anemia; asthma; atherosclerosis; autoimmune vasculitis; cancer; celiac disease; chronic inflammatory demyelinating polyneuropathy (CIDP); chronic obstructive pulmonary disease (COPD); colitis; diverticulitis; endometriosis; familial Mediterranean fever; fatty liver disease; glomerulonephritis; Grave's disease; Guillain-Barre syndrome; Hashimoto's thyroiditis; headaches, including chronic headaches and migraine; hemolytic anemia; hidradenitis suppurativa; HIV and AIDS; hypersensitivity reactions; immune-mediated inflammatory disease (IMID); inflammatory bowel disease such as Crohn's disease and ulcerative colitis; inflammatory myopathies; interstitial cystitis; leukocyte defects; lichen planus; mast cell activation syndrome; mastocytosis; mental health conditions where inflammation and/or autoimmunity is a co-morbid or causative factor, including; depression, schizophrenia, and anxiety; multiple sclerosis; myasthenia gravis; obesity; otitis; pain, including acute and chronic pain; Parkinson's disease; pelvic Inflammatory disorder; peripheral ulcerative keratitis; pernicious anemia; pharmacological inflammatory response; pneumonia; prostatitis; psoriasis; psoriatic arthritis; reperfusion injury; rheumatic fever; rheumatoid arthritis; rhinitis; sarcoidosis; scleroderma; Sjogren's syndrome; systemic lupus erythematosus (SLE); transplant rejection syndrome; type I diabetes; type II diabetes; vasculitis; and vitiligo.

9. A method of treatment comprising administering to a subject in need thereof a therapeutically effective amount of a compound of Formula (I) or Formula (II) or salt thereof.

10. A method of synthesizing a compound of Formula (I) or Formula (II).

11. An intermediate formed in the method of synthesis of the compound of Formula (I) or Formula (II).

12. An intermediate formed in the method of synthesis of the compound of Formula (I) or Formula (II) as claimed in claim 10, wherein the intermediate is a bromine intermediate, as defined by compounds 8 or 9 in Table 1.
